(19) United States
(12) Patent Application Publication
(10) Pub. No.: US 2025/0345120 A1
(43) Pub. Date: Nov. 13, 2025

(54) SURGERY ASSISTING METHODS, SYSTEMS AND DEVICES

(71) Applicant: EIFFEL MEDTECH, Montréal (CA)

(72) Inventor: Frédéric LAVOIE, Montréal (Québec) (CA)

(73) Assignee: EIFFEL MEDTECH, Montréal (CA)

(21) Appl. No.: 19/202,340

(22) Filed: May 8, 2025

Related U.S. Application Data
(60) Provisional application No. 63/644,850, filed on May 9, 2024.

(51) Int. Cl. A61B 34/10; A61B 8/08; A61B 90/00; G06T 5/60; G06T 7/11; G06T 17/00; G16H 30/20

(52) U.S. Cl. CPC A61B 34/10; A61B 90/361; A61B 90/37; G06T 5/60; G06T 7/11; G06T 17/00; G16H 30/20; A61B 8/0875; A61B 2034/105; A61B 2090/367; G06T 2207/10081; G06T 2207/10088; G06T 2207/10132; G06T 2207/20036; G06T 2207/20081; G06T 2207/20084; G06T 2207/30008; G06T 2210/41

(57) ABSTRACT

Methods, devices and systems for planning, monitoring, simulating and/or evaluating a knee surgery procedure. Imaging data of the lower limb is acquired. Interpretable 3D models are generated and combined with static and dynamic parameters into an enhanced bone model. Intervention strategy is simulated. Deviations from the intervention strategy are monitored during surgery. Clinical outcome is predicted from machine learning. Methods for calibrating knee surgery imaging data, establishing an alignment reference frame, and simulating an intervention strategy in a knee surgery procedure.

### CROSS-REFERENCE TO RELATED PATENT APPLICATIONS

This non-provisional patent application claims priority based upon the prior U.S. provisional patent application entitled “Surgery Assisting Methods, systems and devices”, application No. 63/644,850, filed 2024 May 9, in the name of EIFFEL MEDTECH, which is hereby incorporated herein in its entirety.

### TECHNICAL FIELD

The present invention relates to assisting a surgery and, more particularly, to planning, monitoring, and evaluating a surgery.

### BACKGROUND

Medical procedures such as surgery address various conditions that impair limb function and cause patient discomfort. For example, surgery procedures such as a total knee arthroplasty (TKA) may stand out as a prevalent intervention for knee osteoarthritis, aiming to relieve pain and restore joint functionality. Despite its widespread success, a significant percentage of patients report dissatisfaction, leading to considerable socio-economic implications. TKA, along with other surgeries such as tibial or femoral osteotomy, knee ligament reconstruction, and patellar realignment face challenges related to surgical precision and variability in outcomes.

A lack of a standardized reference frame for consistent and reproducible measurements of bone morphology, knee joint alignment, and implant or osteotomy positioning may contribute to the challenges associated with comparing the surgery's outcome. The variance in surgical techniques and the reliance on intraoperative landmark acquisition may introduce biases and uncertainties that compromise the effectiveness of a predictive algorithms and the overall success of surgical interventions.

Current methodologies for preoperative planning and postoperative assessment may also be hindered by incomplete visualization, reliance on bidimensional measurements, patient exposure to radiation, high costs, and procedural complexity. This invention may address some of these limitations.

### SUMMARY

This summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

In a first aspect, the present disclosure relates to devices, systems, and methods for obtaining an interpretable 3D model for a medical procedure on a limb. The devices, systems and methods may further be used for monitoring the medical procedure, predicting a clinical outcome of the medical procedure, training an intervention model, or training an outcome model.

In a second aspect, the present disclosure relates to devices, systems, and methods for monitoring the medical procedure.

In a third aspect, the present disclosure relates to devices, systems, and methods for establishing a measurement reference system for a medical procedure.

In a fourth aspect, the present disclosure relates to devices, systems, and methods for simulating an intervention strategy in a medical procedure on a limb of a patient.

In accordance with the first aspect, a method for obtaining an interpretable 3D model for a medical procedure on a limb is provided. The method includes acquiring imaging data of the limb, reconstructing a reconstructed 3D model of the limb from the imaging data, and calibrating a measurement reference system on the reconstructed 3D model, comprising a set of axes in clinically interpretable anatomical planes, thereby obtaining the interpretable 3D model.

In accordance with the first aspect, a system for obtaining an interpretable 3D model for a medical procedure on a limb is provided. The system comprises an imaging capture module and one or more processors. The imaging capture module may acquire imaging data of a limb. The one or more processors may reconstruct a reconstructed 3D model of the limb from the imaging data and calibrate a measurement reference system on the reconstructed 3D model, comprising a set of axes in clinically interpretable anatomical planes, thereby obtaining the interpretable 3D model. In embodiments, the system may comprise a display device to display reports.

In accordance with the first aspect, a device for obtaining an interpretable 3D model for a medical procedure on a limb is provided. The device comprises one or more processors and a network module for receiving imaging data of the limb. The one or more processors may reconstruct a reconstructed 3D model of the limb from the imaging data and calibrate a measurement reference device on the reconstructed 3D model, comprising a set of axes in clinically interpretable anatomical planes, thereby obtaining the interpretable 3D model. In embodiments, the device may comprise a display module to display reports.

In embodiments, the medical procedure may be a knee surgery procedure, and the limb may be a lower limb. The knee surgery procedure may be one of a knee arthroplasty, a tibial realignment osteotomy, a femoral realignment osteotomy, a knee ligament reconstruction, and a realignment of an extensor mechanism of a knee.

In embodiments, the imaging data may be acquired from at least one of a radiograph, a magnetic resonance imaging (MRI), an ultrasound, and/or a computed tomography (CT) scan.

In embodiments, one or more anatomical landmarks may be detected from the imaging data and the reconstructed 3D model may be reconstructed considering the one or more anatomical landmarks. Optionally, the one or more anatomical landmarks may be detected using one or more of an image segmentation, a linear statistical modeling, a non-linear statistical modeling, and a deep learning technique comprising any one of a convolutional neural network (CNN), a recurrent neural network (RNNs), graph neural networks (GNNs) and a transformer networks. An anatomical landmark of the one or more anatomical landmarks may be represented using a geometric shape comprising a least one of a sphere, a cylinder, a cone, an axis, and a plane.

In embodiments for monitoring the medical procedure, updated imaging data of a current state of the limb may be acquired. The updated imaging data for the measurement reference system may be registered. The interpretable 3D model may be updated with the updated imaging data into an updated 3D model. The updated 3D model may be compared with a planned intervention strategy. Discrepancies may be reported between the current state of the limb and the planned intervention strategy. Optionally, an enhanced bone model may be constructed from the interpretable 3D model and a plurality of measurements from the measurement reference system, and the enhanced bone model may be compared with the planned intervention strategy. Optionally, the imaging data may be updated by integrating an imaging capture module with surgical instruments. Acquiring the updated imaging data may and reporting the discrepancies may be performed in real-time. The discrepancies may be computed by using machine learning algorithms.

In embodiments for predicting a clinical outcome of the medical procedure on a patient, an enhanced bone model may be constructed from the interpretable 3D model and a plurality of measurements from the measurement reference system. One or more intervention strategies may be generated from an intervention scenario and the enhanced bone model. The predicted clinical outcome may be predicted for at least one intervention strategy of the one or more intervention strategies and a plurality of characteristics of the patient. Optionally, the plurality of measurements may comprise at least one of a plurality of morphological parameters, a plurality of alignment parameters and/or a plurality of kinematics parameters. The one or more intervention strategies may comprise at least one of a prosthetic implant positioning, a meniscal repair, a meniscal resection, a patellar resurfacing, a patellar realignment, a cartilage restoration procedure, a tibial realignment osteotomy, a femoral realignment osteotomy, and a reconstruction of one or more ligament. An outcome score of the at least one intervention strategy may be computed, and the predicted clinical outcome may be reported with filtering and sorting according to the outcome score. The predicted clinical outcome may comprise at least one of a likelihood of achieving a mobility threshold, a risk of complications, one or more scores from standardized Patient-Reported Outcome Measures (PROMs), and an overall prognosis for recovery. One or more joint kinematics parameters may be recorded, and the enhanced bone model may be constructed considering the one or more joint kinematics parameters. The one or more joint kinematics parameters may comprise at least one of a rotation, a translation, a weight-bearing gap measurement, a free gap measurement, a manually stressed gap measurement, a mechanically stressed gap measurement, and/or a contact point.

In embodiments, generating the one or more intervention strategies may be performed by using an intervention model trained with machine learning on an intervention training dataset comprising a plurality of training intervention strategies.

In embodiments, the predicted clinical outcome may be predicted using an outcome model trained with machine learning on a clinical outcome training dataset comprising a plurality of training clinical outcomes and a plurality of training characteristics of training patients. Optionally, after a recovery of the patient from the medical procedure, the predicted clinical outcome may be compared to a measured clinical outcome. The measured clinical outcome may be contributed to the clinical outcome training dataset for continuous improvement thereof.

In embodiments, when performed after completion of the medical procedure, an intervention model may be trained. An enhanced bone model may be constructed from the interpretable 3D model and a plurality of measurements from the measurement reference system. The enhanced bone model may be compared to a preoperative enhanced bone model. An executed intervention strategy may be computed from the enhanced bone model and the preoperative enhanced bone model. The executed intervention strategy may be contributed to an intervention training dataset for continuous improvement thereof.

In accordance with the second aspect, a method for calibrating medical imaging data is provided. The method includes accessing a calibration training dataset of calibrated imaging data of a limb, training a calibration model with machine learning on the calibration training dataset and inferring a calibration from an uncalibrated image data of the limb and the calibration model.

In accordance with the second aspect, a system for calibrating medical imaging data is provided. The system comprises one or more processors. The one or more processors may access a calibration training dataset of calibrated imaging data of a limb. The one or more processors may further train a calibration model with machine learning on the calibration training dataset. The one or more processors may further infer a calibration from an uncalibrated image data of the limb and the calibration model.

In accordance with the second aspect, a device for calibrating medical imaging data is provided. The device comprises one or more processors. The one or more processors may access a calibration training dataset of calibrated imaging data of a limb. The one or more processors may further train a calibration model with machine learning on the calibration training dataset. The one or more processors may further infer a calibration from an uncalibrated image data of the limb and the calibration model.

In embodiments, the calibration training dataset may comprise at least one of a radiograph, a magnetic resonance imaging (MRI) image, an ultrasound, and a computed tomography (CT) scan.

In embodiments, the calibrated imaging data may have been calibrated using auto-calibration algorithms.

In embodiments, the calibrated imaging data may be further compensated for geometric distortions and variations in imaging equipment.

In accordance with the third aspect, a method for establishing a measurement reference system for a medical procedure. The method includes acquiring imaging data of a limb, detecting one or more anatomical landmarks within the imaging data, defining the measurement reference system, and applying the measurement reference system to a reconstructed 3D model thereby enabling morphological parameters and alignment parameters measurement. The measurement reference system may be defined from clinically relevant anatomical landmarks of the one or more anatomical landmarks and comprise a set of axes in anatomically interpretable planes.

In accordance with the third aspect, a system for establishing a measurement reference system for a medical procedure. The system comprises an imaging capture module and one or more processors. The imaging capture module may acquire imaging data of a limb. The one or more processors may detect one or more anatomical landmarks within the imaging data. The one or more processors may further define the measurement reference system from clinically relevant anatomical landmarks of the one or more anatomical landmarks, the measurement reference system comprising a set of axes in anatomically interpretable planes. The one or more processors may further apply the measurement reference system to a reconstructed 3D model thereby enabling morphological parameters and alignment parameters measurement.

In accordance with the third aspect, a device for establishing a measurement reference system for a medical procedure. The device comprises one or more processors. The one or more processors may detect one or more anatomical landmarks within imaging data. The one or more processors may further define the measurement reference system from clinically relevant anatomical landmarks of the one or more anatomical landmarks, the measurement reference system comprising a set of axes in anatomically interpretable planes. The one or more processors may further apply the measurement reference system to a reconstructed 3D model thereby enabling morphological parameters and alignment parameters measurement.

In embodiments, the one or more anatomical landmarks may be detected using one or more of an image segmentation, a linear statistical modeling, a non-linear statistical modeling, and a deep learning technique comprising any one of a convolutional neural network (CNN), a recurrent neural network (RNNs), graph neural networks (GNNs) and a transformer networks. A n anatomical landmark of the one or more anatomical landmarks may be represented using a geometric shape comprising a least one of a sphere, a cylinder, a cone, an axis, and a plane.

In accordance with the fourth aspect, a method for simulating an intervention strategy in a medical procedure on a limb of a patient is presented. The method includes, during a training phase of an outcome model, assembling an outcome training dataset, training the outcome model with machine learning on a first subset of the outcome training dataset, and validating an output of the outcome model against a second subset of the outcome training dataset. The method also includes, after the training phase of the outcome model, reconstructing an interpretable 3D model of the limb of the patient from image data acquired prior to executing the intervention strategy and predicting a predicted clinical outcome from the outcome model, the interpretable 3D model, the intervention strategy, an intervention scenario of the intervention strategy, and a plurality of patient metadata. The outcome training dataset may include a plurality of outcome training tuples. Each training tuple may include a training interpretable 3D model of a training limb of a training patient, reconstructed from training image data acquired prior to executing a training intervention strategy, the training interventions strategy, a training intervention scenario of the training intervention strategy, a plurality of training patient metadata comprising demographic, anatomical, and physiological parameters, and a measured clinical outcome observed after the medical procedure.

In accordance with the fourth aspect, a system for simulating an intervention strategy in a medical procedure on a limb of a patient is presented. The system comprises one or more training processors and one or more inferring processors. During a training phase of an outcome model, the one or more training processors may assemble an outcome training dataset comprising a plurality of outcome training tuples. Each training tuples may comprise a training interpretable 3D model of a training limb of a training patient, reconstructed from training image data acquired prior to executing a training intervention strategy, the training intervention strategy, a training intervention scenario of the training intervention strategy, a plurality of train patient metadata comprising demographic, anatomical, and physiological parameters, and a measured clinical outcome observed after the medical procedure. The one or more training processors may further train the outcome model with machine learning on a first subset of the outcome training dataset. The one or more training processors may further validate an output of the outcome model against a second subset of the outcome training dataset. After the training phase of the outcome model, the one or more inferring processors may reconstruct an interpretable 3D model of the limb of the patient from image data acquired prior to executing the intervention strategy. The one or more inferring processors may further predict a predicted clinical outcome from the outcome model, the interpretable 3D model, the intervention strategy, an intervention scenario of the intervention strategy, and a plurality of patient metadata.

In accordance with the fourth aspect, a device for simulating an intervention strategy in a medical procedure on a limb of a patient is presented. The device includes a storage module and one or more inferring processors. The storage module may provide access to an outcome model trained during a training phase by assembling an outcome training dataset comprising a plurality of outcome training tuples. Each training tuples may comprise a training interpretable 3D model of a training limb of a training patient, reconstructed from training image data acquired prior to executing a training intervention strategy, the training intervention strategy, a training intervention scenario of the training intervention strategy, a plurality of train patient metadata comprising demographic, anatomical, and physiological parameters, and a measured clinical outcome observed after the medical procedure. The outcome model may further have been trained with machine learning on a first subset of the outcome training dataset. The outcome model may further have validated a second subset of the outcome training dataset. The one or more inferring processors may reconstruct an interpretable 3D model of the limb of the patient from image data acquired prior to executing the intervention strategy. The one or more inferring processors may further predict a predicted clinical outcome from the outcome model, the interpretable 3D model, the intervention strategy, an intervention scenario of the intervention strategy, and a plurality of patient metadata.

In embodiments, the one or more anatomical landmarks from the outcome training dataset may be detected using one or more of an image segmentation, a linear statistical modeling, a non-linear statistical modeling, and a deep learning technique comprising any one of a convolutional neural network (CNN), a recurrent neural network (RNNs), graph neural networks (GNNs) and a transformer networks. An anatomical landmark of the one or more anatomical landmarks may be represented using a geometric shape comprising a least one of a sphere, a cylinder, a cone, an axis, and a plane.

In embodiments, the one or more intervention strategies may comprise at least one of a prosthetic implant positioning, a meniscal repair, a meniscal resection, a patellar resurfacing, a patellar realignment, a cartilage restoration procedure, a tibial realignment osteotomy, a femoral realignment osteotomy, and a reconstruction of one or more ligament.

### BRIEF DESCRIPTION OF THE DRAWINGS

Further features and exemplary advantages of the present invention will become apparent from the following detailed description, taken in conjunction with the appended drawings, in which:

FIG. 1 is a state diagram depicting an exemplary embodiment of the method for obtaining an interpretable 3D model for a medical procedure on a limb in accordance with the teachings of the present invention;

FIG. 2 is a state diagram depicting an exemplary embodiment of the method for obtaining an interpretable 3D model for a medical procedure on a limb for monitoring the medical procedure in accordance with the teachings of the present invention;

FIG. 3 is a state diagram depicting an exemplary embodiment of the method for obtaining an interpretable 3D model for predicting a clinical outcome of the medical procedure on a patient in accordance with the teachings of the present invention;

FIG. 4 is a state diagram depicting an exemplary embodiment of the method for obtaining an interpretable 3D model for training an intervention model in accordance with the teachings of the present invention;

FIG. 5 is a state diagram depicting an exemplary embodiment of the method for obtaining an interpretable 3D model for training an outcome model in accordance with the teachings of the present invention;

FIG. 6 is a state diagram depicting an exemplary embodiment of the method for calibrating medical imaging data in accordance with the teachings of the present invention;

FIG. 7 is a state diagram depicting an exemplary embodiment of the method for establishing a measurement reference system for a medical procedure in accordance with the teachings of the present invention;

FIG. 8 is a state diagram depicting an exemplary embodiment of the method for simulating an intervention strategy in a medical procedure on a limb of a patient in accordance with the teachings of the present invention; and

FIG. 9 is a logical modular representation of an exemplary system in accordance with the teachings of the present invention;

### DETAILED DESCRIPTION

Traditional methods of preoperative planning for medical procedures such as knee surgeries may not always allow for the precise customization needed for individual patient anatomies. During a medical procedure, unforeseen issues may arise that require on-the-spot adjustments. Predicting postoperative outcomes and recovery trajectories may be challenging with traditional methods, often leading to uncertainties in patient prognosis. The varying anatomy and health conditions of patients require tailored surgical strategies for optimal outcomes. As one example, there is a need for methods, systems and/or devices that incorporate new data and insights into improving surgical techniques and outcomes continually.

The devices, systems and methods presented herein may share common principles worth describing first. Common principles presented hereinbelow should be interpreted as applying to the entire disclosure for similar expressions. Similarly, a description of common expressions presented for one device, system or method may be interpreted as describing similar expressions used for other devices, systems, and methods. Similarly, concepts described for a given method may be interpreted to also apply to a corresponding device or system implementing the given method. Conversely, concepts described for a given device or system implementing a method may also be interpreted as describing the method.

The devices, systems and methods presented herein may be useful for a variety of medical interventions. For example, when the limb is a lower limb, the medical procedure may be interpreted broadly such as to include knee surgery procedures. For illustrative purposes, knee surgery procedures may include knee arthroplasty, a tibial realignment osteotomy, a femoral realignment osteotomy, a knee ligament reconstruction, and a realignment of an extensor mechanism of a knee. Persons skilled in the art will readily recognize that the devices, systems, and methods presented herein may not be limited to knee surgery procedures and may be adapted to include surgery on upper limbs, the thoracic region, the abdomen, the spine, upper extremities, and lower extremities.

The devices, systems, and method presented herein may be used at different moments of a medical procedure. The expression “preoperative” may refer to a period before the medical procedure. Preoperative steps may be performed during a visit of a patient in preparation of the medical procedure, or minutes before the medical procedure begins. Broadly, preoperative steps may be undertaken without direct physical access to a limb. In some circumstances, preoperative steps may also be characterized with access to time consuming equipment or equipment that can generally not be accessed within the vicinity of the medical procedure. The expression “intraoperative” may refer to a period during which the medical procedure is underway. The intraoperative steps may be undertaken while direct access to a limb is enabled through surgery. The intraoperative period may be characterized by time-sensitive operations and an access to equipment within the vicinity of the medical procedure. The expression “postoperative” may refer to a period after the medical procedure has been completed and the limb configuration has been altered. The postoperative steps may be undertaken once the direct physical access to the limb is no longer available. The postoperative period may further be distinguished between a pre-recovery and a post-recovery period. During the pre-recovery period, the limb may not be healed and therefore direct assessment of the outcome may not be achievable. The pre-recovery period may also be characterized by a need for the patient to adapt to the altered limb, for example through physiotherapy. A post-recovery period may describe a period when the limb has healed, at least partially, and the patient has adapted to the altered limb. The post-recovery period may be characterized by an observability of the clinical outcome of the medical procedure.

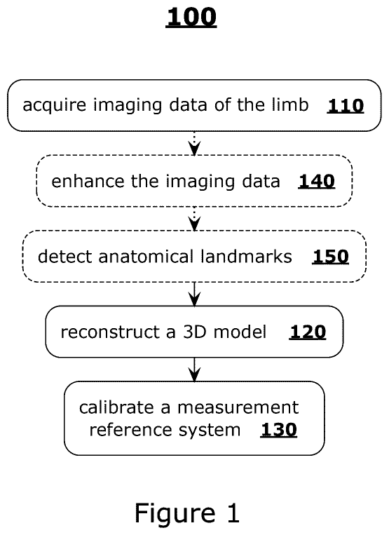

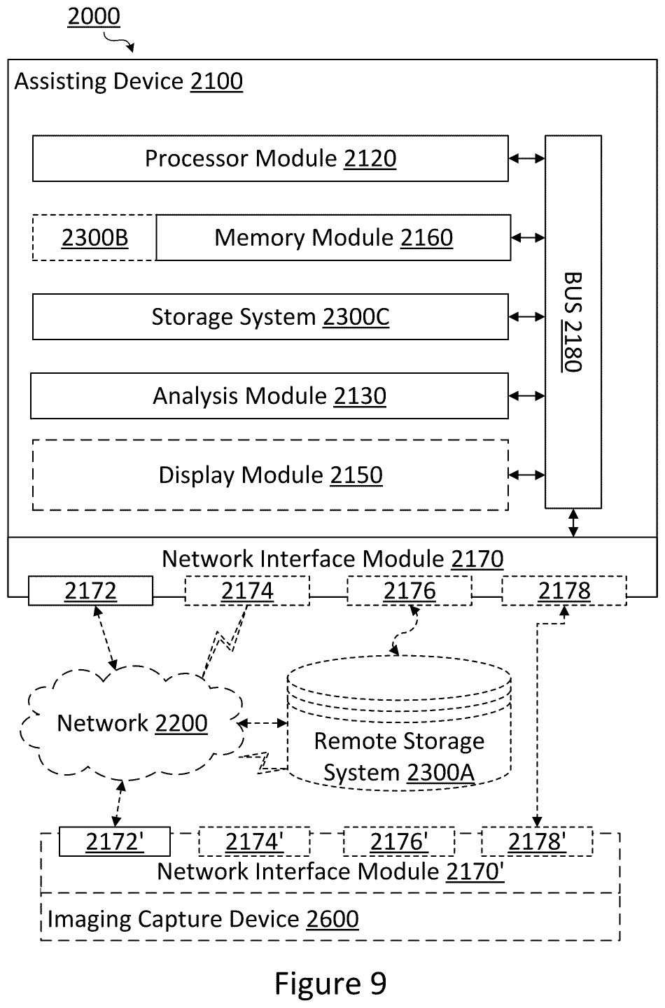

A first aspect of the techniques described herein relates to a method, device, and system for obtaining an interpretable 3D model for a medical procedure on a limb. Reference is now made to the drawings in which FIG. 1 depicts a method 100 for obtaining an interpretable 3D model for a medical procedure on a limb and in which FIG. 9 depicts system 2000 in accordance with the teachings of the present invention.

In embodiments, the medical procedure may be a knee surgery procedure, and the limb may be a lower limb. In embodiments, the knee surgery procedure may be a knee arthroplasty, a tibial realignment osteotomy, a femoral realignment osteotomy, a knee ligament reconstruction, or a realignment of an extensor mechanism of a knee.

The interpretable 3D model may comprise one or many 3D meshes and other properties of the morphology, such as physical properties of tissues and bones and labeled elements, including anatomical landmarks from automatic or manual segmentation. The interpretable 3D model may further comprise texture data from the imaging data acquired and registered onto the 3D meshes. The interpretable 3D model may further comprise a measurement reference system enabling morphological parameters and alignment parameters measurement.

As will be further depicted hereinbelow, the interpretable 3D model may be obtained at different moments of the medical procedure. For example, a preoperative 3D model may be useful in planning the medical procedure, simulating an intervention strategy, and predicting a clinical outcome. Simulating different intervention strategies and evaluating them against training or historical data may offer a way to personalize surgical strategies and may enhance the likelihood of success and patient satisfaction. An intraoperative 3D model may be useful in monitoring the medical procedure and predicting a clinical outcome. During an operation, monitoring an intraoperative 3D model may also enable real-time adjustments, improve surgical outcomes, and may allow for more accurate adjustments during an operation. Given the direct physical access to the limb during the medical procedure, the intraoperative 3D model may also be compared to the preoperative 3D model for improving 3D reconstruction processes. Recording, analyzing, and integrating intraoperative adjustments and postoperative outcomes back into the system may enable continuous learning and improvement of surgery procedures (e.g., knee surgery procedures). A postoperative 3D model may be useful for comparing an executed intervention strategy to a planned intervention strategy for the training of an intervention model. The postoperative 3D model may also be useful for predicting a clinical outcome. Analysis of preoperative and postoperative data using machine learning techniques may enable prediction of outcomes more accurately and may enable better patient counseling and rehabilitation planning.

Imaging data of the limb may be acquired 110. Acquisition 110 of the imaging data may be achieved from imaging data acquisition in a single step or over multiple steps. For example, multiple imaging data acquisition equipment may have been used at different moments and the imaging data of the limb may subsequently be consolidated.

In embodiments, the imaging data may be acquired 110 from at least one of a radiograph, an MRI, an ultrasound, and/or a CT scan. For example, the imaging data may include standard two-dimensional (2D) X-rays used to assess a limb structure and alignment and a limb's overall condition, including the extent of joint degeneration, bone density, and the presence of any fractures or deformities. Weight-bearing X-rays may be useful for assessing the limb under load, offering insights into the functional alignment of the joint. MRI may offer detailed images of both hard and soft tissues, and may be useful for examining the limb's ligaments, tendons, cartilage, and menisci. MRI may reveal subtle injuries or degenerative changes not visible on X-rays. MRI may be useful for planning surgeries that involve soft tissue repair or when assessing the extent of cartilage damage. CT scans may provide high-resolution images of bones and may provide cross-sectional views of the limb. CT scans may be useful for evaluating complex fractures and planning surgeries that require precise bone cutting or reshaping, such as osteotomies. CT scans may also be used to create detailed 3D reconstructions of the knee's bony anatomy. In some cases, ultrasound imaging may be useful for assessing soft tissue structures around the knee, such as tendons and ligaments, in a dynamic, non-invasive manner. Ultrasound imaging may complement MRI or CT findings, especially in evaluating conditions like tendonitis or assessing fluid accumulation in a joint.

The imaging data may be enhanced 140 through one or more image processing techniques. Preprocessing and normalization of the imaging data may enhance the quality of 3D reconstruction. Preprocessing algorithms may improve image clarity and detail. Preprocessing may also include noise reduction, contrast enhancement, and normalization of image intensities. The preprocessing may also highlight and differentiate the relevant anatomical structures (bones, implants, etc.) from the surrounding tissues.

Examples of image processing techniques include an image enhancement model trained with a plurality of enhancement image pairs, each comprising an information poor image and information-rich image. The information poor image may have lower quality, detail, or information content. The information poor image may be blurry, have low contrast, or lack detail due to the limitations of the imaging technology used for capturing. An ultrasound image may for example, capture information poor images. An information-rich image may contain more detail or information and may serve as a reference or target for how the poor-quality image should ideally look. The information-rich image may be obtained using a more advanced imaging technique or under better conditions. The enhancement model may be trained using these pairs of images. By comparing the information-poor images with their information-rich counterparts, the model may learn to identify the enhancements needed to improve the quality of the poor images. The learning process may involve adjusting the model's parameters until reliability is achieved for transformation of poor-quality images into enhanced ones that closely resemble the high-quality reference images. Once trained, the model may be applied to new information-poor images (e.g., ultrasound images obtained during a knee surgery planning process) to enhance the captured images.

A reconstructed 3D model of the limb may be reconstructed 120 from the imaging data. The reconstructed 3D model may include 3D meshes of a three-dimensional representation of a patient's limb, specifically constructed for medical analysis and intervention planning. The reconstructed 3D model may provide a detailed view of the limb's anatomical structure, including bones, soft tissues, and other relevant features. The 3D reconstruction 120 may be performed by mapping the two-dimensional image data onto a three-dimensional space, taking into consideration the spatial relationships and orientations of the anatomical structures captured in the images. Machine learning algorithms may be used to automate and refine the 3D reconstruction process. The resulting reconstructed 3D model may represent the morphology, or the form and structure, of the limb. The structures may include bones, joints, and potentially other relevant structures, depending on the detail and type of imaging data used. When using imaging data captured during the medical procedure, the reconstructed 3D model may reflect the actual conditions during the surgery, offering a real-time view of the patient's anatomy.

In embodiments, one or more anatomical landmarks may be detected 150 from the imaging data and the reconstructed 3D model may be reconstructed 120 considering the one or more anatomical landmarks. The anatomical landmarks may vary according to the anatomical region of the medical procedure. Broadly, anatomical landmarks may be points of interest because they may allow correspondence points across the imaging data, or because they enable positioning and measurements useful for the medical intervention. For example, in the craniofacial region, anatomical landmarks may include the nasion, the orbitale and the gonion. In the thoracic region, anatomical landmark may include the sternal notch and the vertebra prominens. In the abdominal region, anatomical landmarks may include the umbilicus and the anterior superior iliac spine (ASIS). In the spine region, the anatomical landmarks may include the spinous processes and vertebral bodies. In the lower limb regions, the anatomical region may include the greater and lesser trochanters, the epicondyles of the femur and the malleoli. In the hip and thigh region, the anatomical landmarks may include the anterior inferior iliac spine (AIIS), the ischial tuberosity, and the center of the femoral head. In the knee region, the anatomical landmarks may include the patella the tibial tuberosity, the posterior femoral condyles, the tibial plateaus, the tibial intercondylar spines, the intercondylar notch of the femur, and the femoral trochlea. In the leg and ankle region, the anatomical landmarks may include the fibular head and the talar dome. In the shoulder region, the anatomical landmarks may include the acromion and the coracoid process. In the upper limb or arm region, the anatomical landmarks may include the radial head and the olecranon. In the forearm and wrist region, the anatomical landmarks may include the styloid processes of radius and ulna and the lunate bone. In the hand region, the anatomical landmarks may include the metacarpophalangeal joints (MCP) and the distal phalanges. Persons skilled in the art will readily recognize that the anatomical landmarks examples provided herein are not exhaustive. Under most circumstances, the anatomical landmarks may be specific points on the bones or tissues that are easily recognizable and hold clinical significance, such as the femoral epicondyles, tibial tuberosity, or the apex of the patella. Once identified, the specific points of the anatomical landmarks may serve as corresponding points across multiple images from the imaging data, enabling the reconstruction 120 of the reconstructed 3D model.

The one or more anatomical landmarks may be detected 150 using one or more of an image segmentation, a linear statistical modeling, a non-linear statistical modeling, and a deep learning technique comprising any one of a convolutional neural network (CNN), a recurrent neural network (RNNs), graph neural networks (GNNs) and a transformer networks.

An image segmentation may be used to divide a digital image into multiple segments (sets of pixels, also known as image objects) to simplify representation. The segmentation may allow simpler analysis (e.g., less resource-intensive computing) and/or may augment the objective value of the resulting analysis (e.g., improved image objects matching). Image segmentation may partition an image into regions that have a strong correlation with objects or areas of interest in the real world. The regions may be selected based on predetermined criteria such as color, intensity, texture, or other visual characteristics inherent to the image. The outcome of image segmentation may be a collection of segments that collectively cover the entire image, or a set of contours extracted from the image. Segmentation may divide image data into segments or regions that correspond to different anatomical structures. Segmentation may be performed manually by experts who delineate structures of interest, or automatically using algorithms that identify boundaries based on differences in texture, color, or intensity. For example, each of the pixels in a region may be similar with respect to some characteristic or computed property, such as color, intensity, or texture. Image segmentation may also include thresholding, edge-based segmentation, region-based segmentation, clustering, and watershed segmentation.

Linear statistical modeling for 3D model reconstruction may assume a linear relationship between input data (e.g., image features) and output variables (e.g., 3D shape parameters). Linear models may be effective for image processing where relationships between variables are proportional and additive. Linear models may be used for tasks such as image correction (e.g., adjusting brightness and contrast) and feature extraction where the relationship between variables is straightforward and proportional. Linear models may for example help correct systematic artifacts in images, such as those caused by the imaging equipment or by predictable patient movements, by applying inverse transformations that counteract these effects. Image registration may align and stitch together images from different angles or at slightly different times (e.g., from a series of CT slices) using linear transformations to rotate, scale, and translate images so they fit together accurately. Linear statistical modeling may infer the 3D structure of an object or scene from one or more 2D images, relying on statistical correlations between observed image data and known 3D shapes. Examples of linear statistical modeling in 3D model reconstruction may include principal component analysis (PCA)-based shape modeling, linear shape from shading, multiview stereo reconstruction with linear calibration, linear photometric stereo, and linear regression models for depth estimation.

Non-linear models may be necessary when the relationships between variables in the image data are complex and not well-represented by straight lines or simple additive effects. Non-linear models may be useful for enhancing image features and segmentation, for example. Deep learning models may synthesize the outputs of linear and non-linear statistical models, incorporating corrections, enhancements, and segmentations into a cohesive whole. Non-linear statistical models may, for example, include non-linear shape from shading, texture, silhouette, motion, focus/defocus. Non-linear statistical models may also include 3D reconstruction from point clouds with non-linear optimization, deep learning-based 3D reconstruction, non-linear manifold learning for 3D shape analysis, and non-linear regression for depth estimation.

The detection 150 of anatomical landmarks and the 3D reconstruction 120 may also be performed through deep learning technique comprising. For example, convolutional neural network (CNN) may be particularly well-suited for processing grid-like data, such as 2D images or 3D image volumes. In 3D model reconstruction, CNNs may analyze slices of medical images (e.g., from MRI, CT scans) to identify features and patterns relevant to the structure of interest. Through techniques like volumetric segmentation, CNN s may delineate the boundaries of organs or lesions, enabling the reconstruction of the 3D models. CNNs may be particularly well suited for capturing spatial hierarchies in images such as shapes and textures specific to various tissues and structures. Recurrent neural network (RNNs) may be designed to handle sequential data. For 3D model reconstruction, RNNs may analyze sequences of 2D image slices, capturing the spatial relationships and continuity across slices. Sequential processing may enable the reconstruction of complex 3D structures by aggregating information across the sequence of images. RNNs may be particularly suited for dealing with the order and context in sequences, which may be beneficial when the spatial order of slices influences the 3D structure. Graph neural networks (GNNs) may model the relationships between different regions or points within medical images as a graph where nodes represent regions or keypoints, and edges represent spatial or anatomical relationships. GNNs may be well suited for reconstructing the 3D geometry of complex, irregular connectivity patterns inherent in biological and anatomical structures. Transformer networks may be well suited for handling long-range dependencies in data. For 3D reconstruction, transformers may process an imaging dataset in parallel, capturing global context and dependencies between distant regions. Transformers may be particularly well suited for tasks requiring integration of information across extensive spatial or temporal domains.

For deep learning techniques, the images may initially be manually annotated in the dataset by marking the relevant anatomical landmarks. The manual marking process may be performed by experts in anatomy and to identify and label these anatomical landmarks on the images. The annotations may be used to create a training dataset pairing the images with the corresponding annotations of anatomical landmarks and serving as a ground truth for training the machine learning model.

While several machine learning techniques may be able to train on the dataset, CNN s may be particularly well suited for anatomical landmark tasks. During training, the model may learn to recognize the patterns in the images that correspond to the annotated anatomical landmarks. The trained model may be validated using a separate set of images not seen by the model during training. Validation may assess the model's accuracy in identifying and mapping the anatomical landmarks. Once trained, new surgery imaging data may be processed through the model to automatically identify and map the anatomical landmarks in real-time or near-real-time. CNNs may be trained to recognize and pinpoint anatomical landmarks in the images. Multiple images may therefore be aligned, and the orientation of the bone structures may be determined.

In the case of ultrasound, despite limitations in providing clear bone images, artificial intelligence (AI) models may be trained to identify anatomical landmarks on soft tissue-bone interfaces or specific patterns indicative of bone surface positions. Using deep learning techniques, such as CNNs, the images may be segmented to differentiate bone tissue from other tissues and artifacts. Segmentation may enable extraction of the bone structure from the images accurately. Segmented images and identified anatomical landmarks may be used together for 3D reconstruction algorithms through techniques such as volumetric reconstruction for CT and MRI images, where the segmented slices are compiled into a 3D volume. For ultrasound, AI techniques may be used to infer the 3D positioning of bone surfaces from 2D images. UItrasounds, during a medical procedure, may also be used for alignment of preoperative 3D models with the patient's actual bone structure. For example, alignment may be achieved through the identification of bone contours and by an ultrasound probe that is outfitted with positional sensors. Ultrasounds may be used for rigid registration of the 3D models within the surgical navigation volume. The registration aligns preoperative 3D models with the patient's anatomy.

An anatomical landmark of the one or more anatomical landmarks may be represented using a geometric shape comprising a least one of a sphere, a cylinder, a cone, an axis, and a plane. Geometric methods may be used to configure basic geometric shapes—such as spheres, cylinders, cones, along with axes and planes—to accurately represent, approximate, or highlight key anatomical features within medical images or 3D reconstructions. For example, a sphere may be employed to model a tumor, with its center determined by calculating the centroid of the boundary points detected through image segmentation, and its diameter established by the average distance from this centroid to the points on the boundary. Similarly, a cylinder or cone may be used to represent elongated structures like bones or vessels, where the axis of these shapes aligns with the longitudinal orientation of the structure, and the dimensions (radius and height) are calculated to match the structure's size. Axes and planes may be used for defining orientations and spatial relationships between different anatomical landmarks. An axis might represent the alignment of a limb, aiding in understanding joint articulations, while a plane may be used to delineate the boundary between different tissue types or to guide the direction of surgical incisions. The mapping of the geometric shapes and constructs onto the anatomical landmarks may be achieved through image processing and geometric fitting algorithms. The algorithms may, for example, analyze the contours and features identified in the medical images to best fit the selected geometric shape, taking into account the spatial properties and orientations that are characteristic of the anatomical structure being modeled.

The reconstructed 3D model may be refined to improve accuracy and detail. Refinement may include smoothing algorithms, manual corrections based on expert knowledge, or additional AI-assisted enhancements. The accuracy of the reconstructed 3D model may be validated against known anatomical structures or through comparison with other reconstructed 3D models generated for other patients. Validated samples may be used in conjunction with supervised deep learning to create models trained to compute reliable reconstructed 3D models from labeled 2D images.

A measurement reference system may be calibrated 130 on the reconstructed 3D model thereby obtaining the interpretable 3D model. The measurement reference system may comprise a set of axes in clinically interpretable anatomical planes. The measurement reference system may be a framework, or a set of guidelines used to make precise and consistent measurements across the interpretable 3D model. When multiple interpretable 3D models are used to represent a limb, individual reference systems may be provided in addition or in replacement of a common measurement reference system. The axes and planes of the reference system may provide a standardized way to describe the positions and movements of different parts of the limb. The anatomical planes may include a sagittal plane, a coronal (Frontal) and a transverse (Axial) plane. The sagittal plane may divide the body into right and left parts or an anatomical structure in medial and lateral parts. The coronal plan may divide the body into front (anterior) and back (posterior) parts. The transverse plane may divide the body into upper (superior, or proximal) and lower (inferior or distal) parts. Using clinically interpretable axes, the outcomes of these measurements may be understood and applied by medical professionals in the context of diagnosing conditions, planning surgeries, and predicting surgical outcomes. For some medical procedures, additional or alternative reference systems may be employed. For example, in dentistry and orthodontics, the occlusal plane may be used as a reference plane. In podiatry, the midtarsal plane and the metatarsal plane may be used as reference planes. In hand surgery, the hand and wrist may use specific reference points and planes for precision, such as the midcarpal plane.

The interpretable 3D model, now enriched with dimensional and morphological parameters, may be used to measure distances and orientation of bones, ligaments, and tissues of the interpretable 3D model. The measurement reference system may allow for the standardized measurement of morphological parameters (e.g., bone lengths, widths, curvatures), alignment parameters (e.g., angles and orientations of bones and joints), and possibly kinematics parameters (e.g., ranges of motion, mechanical behaviors under stress) within the interpretable 3D model.

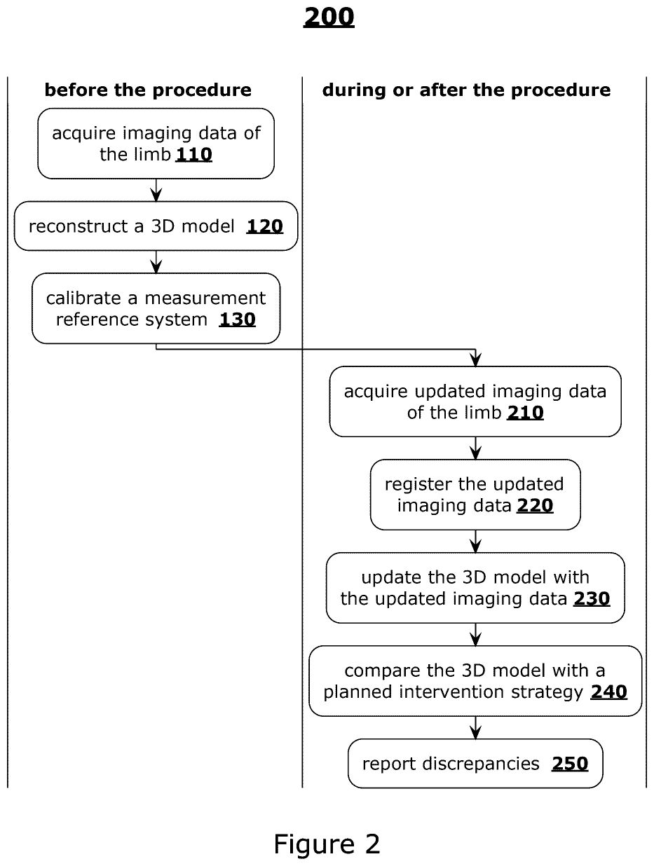

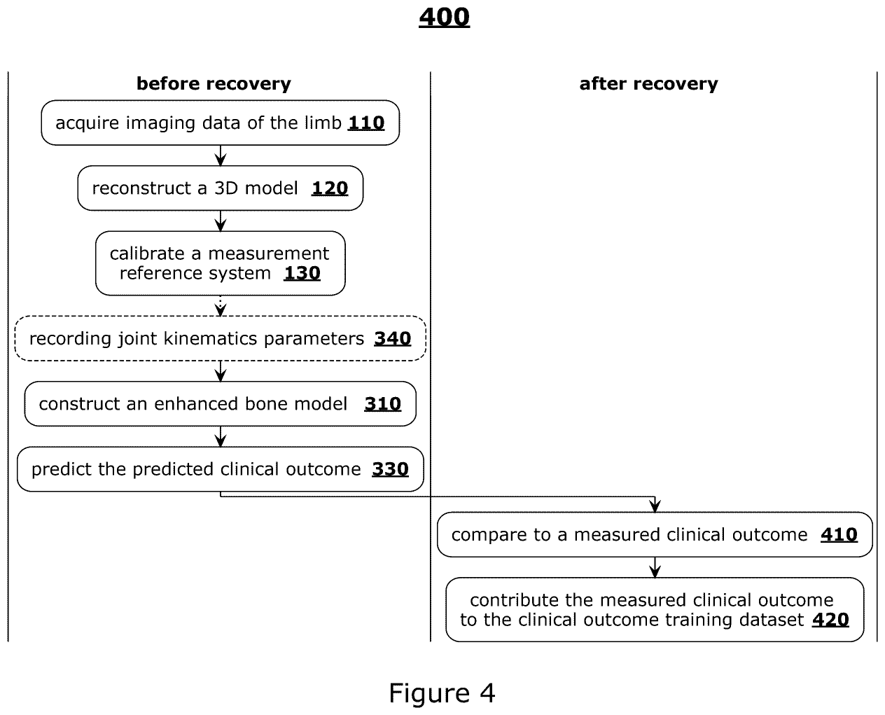

An interpretable 3D model of a limb may be obtained for monitoring the medical procedure. Reference is now made to the drawings in which FIG. 2 depicts a method 200 for obtaining an interpretable 3D model for a medical procedure on a limb for monitoring the medical procedure in accordance with the teachings of the present invention. FIG. 4 depicts a method 400 for obtaining an interpretable 3D model for training an outcome model.

Imaging data of a current state of the limb may be acquired 210. Acquiring 210 the updated imaging data of a current state of the limb during a medical procedure may involve real-time or near-real-time imaging technologies such as ultrasound, fluoroscopy, intraoperative computed tomography (CT) or optical imaging systems. Ultrasound imaging may obtain real-time images of soft tissue structures. Fluoroscopy may use continuous X-ray beams to produce real-time video images of internal structures. Intraoperative CT scanners may provide high-resolution cross-sectional images of bones during surgery.

The imaging data may be updated by integrating an imaging capture module with surgical instruments. Optical imaging systems, including those based on structured light or laser technologies, may capture the surface geometry of exposed tissues. When used in surgery. Some surgical instruments may also be equipped with sensors that may provide real-time data on their positioning relative to the anatomy. For instance, navigation systems used in orthopedic surgery may track the position of cutting tools and implants with respect to the bones, assisting surgeons in maintaining alignment and orientation according to the preoperative plan.

The updated imaging data may be registered 220 for the measurement reference system. Registration 220 may refer to the process of aligning the updated imaging data with the measurement reference system defined for the interpretable 3D model. The measurement reference system may consist of a set of axes and planes that are defined based on anatomically significant landmarks on the limb being operated on. Imaging data of the limb acquired 210 during surgery may need to be aligned (or “registered”) with the measurement reference system to ensure that any measurements, adjustments, or analyses are accurate and consistent with other aspects of the interpretable 3D model. For registration purposes, embodiments may rely on optical or electromagnetic tracking devices to monitor the position and orientation of surgical instruments in real-time. Tracking data may be combined with the imaging data to enhance the accuracy of registration enabling the updated 3D model to accurately reflects the current state of the limb.

The interpretable 3D model may be updated 230 with the updated imaging data into an updated 3D model. Once the updated imaging data is registered to the measurement reference system, the imaging data may be incorporated into the interpretable 3D model of the limb to obtain an updated 3D model. Incorporation of the updated imaging data to the interpretable 3D model may enable the updated 3D model to reflect any changes in the anatomy or surgical modifications that have occurred since the last imaging session. In some cases, manual input such as annotations or corrections may be used to fine-tune the registration of imaging data and updates to the interpretable 3D model into the updated 3D model.

The updated 3D model may be compared 240 with a planned intervention strategy. Before surgery, a planned intervention strategy may be formulated based on the patient's diagnostics, medical history, anatomy, and the specific goals of the surgery. The planned intervention strategy may outline the surgical steps, desired adjustments, or corrections (such as realigning bones or placing implants), and the expected anatomical outcomes. The planned intervention strategy may include in some cases, placement of an implant, expected alignment corrections, and anticipated kinematics. The planned intervention strategy may be visualized in the form of a rendering of the interpretable 3D model or simulation that may predict the postoperative state of the limb. The planned intervention strategy may be an intervention strategy obtained from an intervention model such as the one trained with method 400 further presented hereinbelow.

The comparison 240 may be achieved by overlaying or juxtaposing the updated 3D model with the 3D representation of the planned surgical outcome. The comparison may allow the surgical team to assess how closely the intraoperative modifications match with the planned intervention strategy. Discrepancies and deviations from the intended outcomes may be identified, providing an opportunity for real-time adjustments.

In embodiments, an enhanced bone model may be constructed from the interpretable 3D model and a plurality of measurements from the measurement reference system, and the enhanced bone model may be compared with the planned intervention strategy. The enhanced bone model may be defined and obtained as further described in method 300 as will be more fully described hereinbelow. Broadly, the enhanced bone model may be the interpretable 3D model enriched with additional parameters to enable simulation of intervention strategies. The additional parameters may include static and dynamic parameters and may be measured from the interpretable 3D model or directly on the limb. For example, during a knee surgery, joint kinematics parameters, such as the range of motion, stability in different planes, and the tracking of the patella may be recorded. The joint kinematics parameters may include a rotation in three planes, a translation in three planes, a gap measurement during full range of motion with application of stress, a gap measurement during full range of motion without application of stress, a contact point, and/or a patellar tracking. Kinematics parameters monitoring may be performed through manual manipulation of the joint by the surgeon and the limb's position and movements may be tracked using the imaging system and sensors. The recorded parameters may be compared with the expected kinematics outlined in a preoperative enhanced bone model used to generate the planned intervention strategy. Discrepancies may indicate areas that require further adjustment to achieve the desired surgical outcome.

Discrepancies may be reported 250 between the current state of the limb and the planned intervention strategy. The comparison may be both visual, allowing surgeons to see the differences between the current state of the limb and the planned intervention strategy, and quantitative, providing metrics such as distances, angles, and volumes that indicate the extent of deviation from the planned intervention strategy. For example, when an implant is not correctly aligned according to the planned intervention strategy, the degree and direction of misalignment may be indicated.

Acquiring 210 the updated imaging data and reporting 250 discrepancies may be performed in real-time. Real-time may be interpreted in the current context as being within a short delay such that the feedback from the image may be useful during the operation, without causing undue delay. For example, capture of the updated imaging data leading to a display of the image within 500 ms would typically be considered real-time, while a display within 2 seconds may be considered semi-realtime. Ultrasound may be used for real-time imaging due to its non-invasive nature and ability to provide images without radiation. Ultrasound may provide detailed views of soft tissues and may even guide the placement of instruments or implants during surgery. Fluoroscopy may provide continuous X-ray imaging and may be useful for visualizing bones and the placement of metallic implants. Real-time fluoroscopic data may be integrated into the updated 3D model to check alignment and implant positioning. Mobile C-arm X-ray machines, optical imaging systems and, in some cases, intraoperative CT (iCT) images of the surgical may also provide real-time imaging data during the medical procedure. Once captured, images may undergo immediate processing to enhance quality, remove noise, and highlight relevant features. Techniques such as segmentation may be used to isolate areas of interest, such as the bones and surrounding tissues. Convolutional Neural Networks (CNNs) and other techniques previously described hereinabove may also be used to automate and enhance the recognition of anatomical landmarks and the segmentation of images.

The discrepancies may be computed by using machine learning algorithms. A discrepancy training dataset comprising preoperative interpretable 3D models, detailed intervention strategies, and corresponding intraoperative or postoperative updated 3D models may be collected. The discrepancy training dataset may also include outcomes indicating whether the intervention achieved pre-established goals or if discrepancies led to adjustments and/or suboptimal results. Relevant features from both preoperative and intraoperative models that contribute to successful outcomes may be identified and extracted. Features might include specific anatomical landmarks, spatial relationships between structures, intended versus actual implant positions, and other geometric or morphological attributes. A discrepancy model may be trained on the discrepancy training dataset to recognize patterns that represent a successful alignment between preoperative plans and intraoperative/postoperative states, as well as patterns indicative of discrepancies. Supervised learning techniques, such as classification or regression algorithms, may be used, where the output may be the presence, type, and magnitude of discrepancies. During surgery, when an updated 3D model is obtained, the same set of features that were identified as relevant during the training phase may be extracted. The extracted features may be used against the trained discrepancy model to analyze the current state in comparison to the preoperative plan. The discrepancy model may apply the trained patterns to identify any relevant deviations from the planned intervention strategy, such as misalignments, incorrect positioning of implants, or other unforeseen changes in the surgical site.

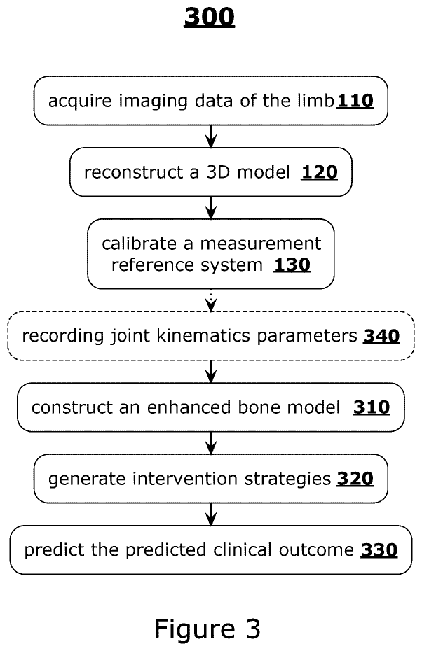

An interpretable 3D model of a limb may be obtained for predicting a clinical outcome of the medical procedure on a patient. Reference is now made to the drawings in which FIG. 3 depicts a method 300 for obtaining an interpretable 3D model for a medical procedure on a limb for predicting a clinical outcome of the medical procedure on a patient in accordance with the teachings of the present invention.

An enhanced bone model may be constructed 310 from the interpretable 3D model and a plurality of measurements from the measurement reference system. The enhanced bone model may also be enhanced with additional data layers or annotations that may be useful for surgical planning, such as identifying potential osteotomy sites or optimal locations for implant placement. The enhanced bone model may be used to assess the anatomy of a patient's limb in a standardized and clinically meaningful way, simulate surgical interventions, including adjustments to bone structures and placements of prosthetic components and predict the clinical outcome of different surgical strategies by comparing various options within the preoperative enhanced bone model.

For some medical procedures, multiple enhanced bone models may be constructed 310 from one or several interpretable 3D models. The expression “enhanced bone model” may be interpreted broadly to encompass models that may comprise more than one bone, such as tissues, ligaments, tendons, and cartilage. The same interpretable 3D model may be used to generate more than one enhanced bone model. For examples, different enhanced bone models from the same interpretable 3D model may include different measurements for different purposes. The interpretable 3D model may include precise information about the anatomy of the limb, including bone structures, joint alignments, and the spatial relationships between different anatomical features and may be created from the teachings of method 100 hereinabove.

The enhanced bone model, whether preoperative, intraoperative, or postoperative may be described as rich virtual representations of the lower limb representative of the known state of the lower limb before, during and after a knee surgery, respectively. Each representation may comprise more than just bone structures. For example, an enhanced bone model may comprise a system of soft and hard tissues represented by the interpretable 3D models and imaging data, existing or planned prosthetic components, in addition to static and dynamic parameters describing the anatomical system. Broadly, the enhanced bone model may be used to simulate intervention strategies such as prosthetic positioning and predict clinical outcomes. The data used to compute an enhanced bone model may not be limited in time. For example, an intraoperative or postoperative enhanced bone model may comprise preoperative data and even patient data from files.

The measurements may include morphological parameters, alignment parameters and/or kinematics parameters. Broadly, the measurements may include static parameters such as morphological parameters and alignment parameters, and dynamic parameters such as kinematics parameters.

Static parameters may refer to the anatomical and geometrical features of the enhanced bone model that do not change with movement, excluding the small-scale stress-induced deformations of bones. Static parameters may be measured in a stationary position and may include bone geometry, spatial relationships, joint angles, bone density and anatomical landmarks. For example, for a knee surgery, bone geometry may include the shapes, sizes, and contours of the bones and may, for example, includes the curvature of the femoral condyles, the tibial plateau's slope, and the thickness of the patella. The spatial relationships may include the relative positions of bones to one another, such as the alignment of the femur and tibia, which may influence knee joint stability and load distribution. Joint angles may include angles at which bones meet to form joints, such as the varus (inward angulation) or valgus (outward angulation) alignment of the knee. The bone density may indicate stress distribution within the knee joint. Anatomical landmarks may represent key points used for orientation and measurement, including the femoral epicondyles, tibial tuberosity, and the apex of the patella. Specifically, morphological parameters may be used to calculate and extract specific static parameters such as such as bone curvatures, joint angles, and lengths in reference to the measurement reference system. Alignment parameters may be used to calculate and extract specific static parameters such as such as varus or valgus alignment in reference to the measurement reference system. Alignment parameters may be determined based on the positions and orientations of the bones relative to each other and to predefined anatomical axes. Angles may be calculated and distances between different anatomical landmarks may be measured to assess the overall alignment of the lower limb. Parameters such as varus/valgus angles, tibial slope, and mechanical axis deviation may be examples of alignment metrics that may be derived from the interpretable 3D model.

Dynamic parameters may capture the functionality and behavior of the knee joint during movement. Dynamic parameters may include range of motion (ROM), kinematics, joint loading, ligament tensions, and muscle forces, for example. ROM may include the degrees through which the knee can flex (bend) and extend (straighten). In the case of a knee surgery, for example, kinematics parameters may describe the motion of the knee joint, including the path of the tibia relative to the femur during flexion and extension, and the tracking of the patella through the femoral groove. Joint loading may include the distribution of forces across the knee joint during different activities (walking, climbing stairs), which affects wear patterns and the longevity of implants. Ligament tensions may include the stretching or relaxation of ligaments at various angles of knee flexion, which contributes to joint stability and influence surgical decisions, especially in ligament repair or reconstruction. Muscle forces may include the forces exerted by muscles surrounding the knee, which may impact joint mechanics and may guide rehabilitation strategies post-surgery.

One or more joint kinematics parameters may be recorded 340, and the enhanced bone model may be constructed 310 considering the one or more joint kinematics parameters. The joint kinematics parameters may comprise at least one of a rotation, a translation, a weight-bearing gap measurement, a free gap measurement, a manually stressed gap measurement, a mechanically stressed gap measurement, and/or a contact point. The one or more joint kinematics parameters may comprise at least one of a rotation, a translation, a weight-bearing gap measurement, a free gap measurement, a manually stressed gap measurement, a mechanically stressed gap measurement, and/or a contact point. Kinematics parameters may be used to calculate and extract specific dynamic parameters describing the potential movements of the bones based on the measurement reference system.

One or more intervention strategies may be generated 320 from an intervention scenario and the enhanced bone model. The intervention strategies may refer to detailed specifications regarding how surgical interventions, such as the placement of implants or the alignment of bones, should be conducted. In embodiments, generating the one or more intervention strategies may be performed by using an intervention model trained with machine learning on an intervention training dataset comprising a plurality of training intervention strategies. An intervention strategy may refer to a hypothetical, planned or executed course of action designed to address a specific medical condition or achieve a particular treatment objective. An intervention scenario may include the objective of the intervention, the type of medical procedure, the surgical techniques and approaches, the implant or graft selection, and the expected modifications to anatomy. For example, an intervention scenario may include constraints related to the desired goals and limitations of the surgery, such as improving joint mobility, minimizing pain, or ensuring the long-term viability of an implant. The constraints may be determined based on clinical best practices, patient health considerations, and the specific objectives of the surgery. Patient-specific metadata may encompass a wide range of information unique to the patient, including demographic details (age, sex, etc.), physiological data (e.g., bone density, muscle strength), and medical history (previous surgeries, underlying conditions). The metadata may provide a comprehensive profile of the patient that can influence surgical planning and outcomes.

The intervention strategy may include a prosthetic implant positioning, a meniscal repair, a meniscal resection, a patellar resurfacing, a patellar realignment, a cartilage restoration procedure, a tibial realignment osteotomy, a femoral realignment osteotomy, and a reconstruction of one or more ligament. The intervention scenarios may be adapted for each type of medical procedure. The intervention scenarios provide context for the medical procedure, including an objective, characteristics of a patient and constraints. Multiple intervention strategies may be elaborated for a given intervention scenario.

An intervention strategy may detail how a medical procedure, such as a knee surgery, may be conducted. The intervention strategy may include decisions about surgical techniques, the selection and placement of implants (if applicable), modifications to the bone structure, and any additional procedures. The intervention strategy may be customized for specific needs of a patient, based on the interpretable 3D model generated from imaging data and considering the desired intervention scenario (the goal of the surgery, such as improving mobility or reducing pain).

A clinical outcome may be predicted 330 for at least one intervention strategy of the one or more intervention strategies and a plurality of characteristics of the patient. The predicted clinical outcome may comprise at least one of a likelihood of achieving a mobility threshold, a risk of complications, one or more scores from standardized Patient-Reported Outcome Measures (PROMs), and an overall prognosis for recovery. The morphological and alignment parameters from the calibrated enhanced bone model and the enhanced bone model may be consolidated with additional patient data that may influence recovery outcomes. The additional patient data may include patient demographics, preoperative health status, surgical details, and any specific interventions performed. A predictive outcome model may be trained using historical datasets that include similar enhanced bone models and known outcomes. The historical outcome datasets may provide examples of how various factors have influenced recovery trajectories, mobility achievements, complication rates, and overall prognoses in past patients. Machine learning algorithms, such as regression models, decision trees, or neural networks, may be applied to identify patterns and relationships within the data as further presented in method 800, which will be more fully described hereinbelow.

The planned surgical intervention may be simulated within the outcome model. When postoperative imaging data is not yet available, the simulation may adjust the interpretable 3D model and enhanced bone model based on the interventions strategy to reflect the expected post-surgical anatomy, incorporating changes in bone geometry, implant placement, and altered soft tissue tensions. From the interpretable 3D model, biomechanical modeling may simulate the forces and movements within the knee joint to understand how the reconstructed knee will respond to loads and movements. The modeling may consider interactions between different knee components (e.g., how an implant might alter load distribution across the joint surface) and simulates a range of motions (flexion, extension, rotation, etc.) to assess functionality. Machine learning algorithms may be used for immediate identification of discrepancies between a planned intervention strategy and an actual intervention strategy or analyze the biomechanical model and predict the kinematics of the knee post-surgery and be used to create an adjusted enhanced bone model. The outcome models may be trained on datasets of previous surgeries, incorporating outcomes and the whole or a subset of the enhanced bone model attributes, to identify patterns and correlations. The outcome models may consider the biomechanical data along with patient-specific factors to predict how the knee may move, how stable it may be in different positions, and potential ranges of motion after recovery.

An outcome score of the at least one intervention strategy may be computed. The predicted clinical outcome may be reported with filtering and sorting according to the outcome score. An outcome score of an intervention strategy of the one or more intervention strategies may be computed, and the predicted clinical outcome may be reported with filtering and sorting according to the outcome score. The outcome score may serve as a quantifiable metric designed to predict the effectiveness or success of a given intervention strategy in achieving desired clinical outcomes, such as improved mobility, reduced pain, or minimized risk of complications. The predicted clinical outcomes may be organized based on the computed outcome scores allowing for filtering and sorting of the intervention strategies and enabling healthcare professionals to compare and select the most appropriate surgical approach.

In embodiments, multiple predicted clinical outcomes may be predicted 330, each with a specific purpose. The report may be designed to assist in surgical planning by providing a recommendation based on an analysis of patient-specific data and predictive outcome modeling. The recommended intervention strategy within the enhanced bone model may outline the specific surgical actions suggested by the outcome model, visualized within the context of the patient's interpretable 3D model, and providing a visual and descriptive guide to how the surgery may be conducted to achieve the best outcomes. For instance, for a patient undergoing knee arthroplasty, a report might visualize and describe the optimal size, type, and positioning of the knee implant within the interpretable 3D model of the patient's knee. The report may specify angles of insertion and depths that are predicted to offer the best functional outcome based on the patient's unique anatomy. The predicted clinical outcomes section of the report may forecast the expected results of the surgery based on the recommended intervention strategy. The predictions may consider the patient's specific conditions and the collective knowledge learned from previous similar cases by the outcome model. For example, the report may predict improvements in knee function, such as an increase in the range of motion from 60 degrees pre-surgery to 120 degrees post-surgery. The report might also predict the level of pain reduction using a standard pain scale or the likelihood of returning to certain activities without discomfort. The confidence score indicating a likelihood of achieving the predicted clinical outcomes may be a quantitative measure, possibly expressed as a percentage, reflecting the outcome model's certainty regarding the predicted clinical outcomes. For example, if the report predicts a successful recovery with improved mobility and reduced pain, the report may also provide a confidence score of 85%, suggesting a high level of confidence in these outcomes based on the model's analysis.

In embodiments, generating 320 the one or more intervention strategies may be performed by using an intervention model trained with machine learning on an intervention training dataset comprising a plurality of training intervention strategies. The computation of an outcome score for an intervention strategy in knee surgeries may leverage machine learning models trained on historical clinical outcomes and patient characteristics. The exact mathematical model and algorithmic specifics may vary based on the data and outcomes prioritized, the general process may involve collecting a comprehensive dataset comprising historical intervention strategies, patient characteristics (age, body mass index, pre-existing conditions), surgical outcomes (mobility, pain levels, complication rates), and patient-reported outcome measures (PROMs). Features from the dataset that are most predictive of surgical outcomes may be identified and selected. The selection may include specific details about the intervention (type of surgery, implants used, specific surgical steps), as well as patient-specific data. Features may be chosen based on their relevance and statistical significance in affecting the outcomes. The selected features may be used to train a machine learning model. The model may be trained to predict the outcome of a surgery based on the intervention strategy and patient characteristics. Typical machine learning algorithms for such tasks may include regression models, decision trees, random forests, or neural networks. The choice of algorithm may depend on the complexity of the data and the specific outcomes being predicted. Once the model is trained, it may compute an outcome score for any given intervention strategy by evaluating the predicted effectiveness of the strategy in achieving desired clinical outcomes. The score may quantify of the predicted success, based on the historical data and the model's learning. For example, a score may predict the success of knee arthroplasty in improving patient mobility, with success measured on a scale from 0 to 100 (where 100 represents full mobility without complications). A dataset may be compiled from past knee arthroplasty surgeries, including patient age, body mass index (BMI), type of implant used, surgical technique, and patient-reported mobility levels post-surgery. Analysis may reveal that patient BMI, type of implant, and surgical technique are significant predictors of mobility outcomes. A regression model may be trained using the selected features. The model may learn the relationship between the features and post-surgery mobility scores. The trained model may use the input data to predict a mobility score of 85 for this specific combination of intervention strategy and patient characteristics. A computed outcome score of 85 may suggest a high likelihood of achieving successful mobility post-surgery for this patient, based on the historical data and the patterns learned by the model. The score may then be used to filter and sort potential intervention strategies.

Predicting 330 a clinical outcome of the medical procedure on a patient may be performed at different moments with useful purposes. When performed against preoperative imaging data and a preoperative enhanced bone model, the method may be used to score different intervention strategy for a given intervention scenario and select one that is most appropriate for a patent according based on specific characteristics of the patient. When performed against intraoperative imaging data, the predicted clinical outcome may be useful for comparing different intervention strategies based on the actual state of the limb, as directly visible during the intervention. When performed against postoperative imaging data, the predicted clinical outcome may be useful to establish a recovery plan and manage the patient's expectations.

An interpretable 3D model of a limb may for training an outcome model. Reference is now made to the drawings in which FIG. 4 depicts a method 400 for obtaining an interpretable 3D model for training an outcome model in accordance with the teachings of the present invention.

A predicted clinical outcome may be predicted 330 using an outcome model trained with machine learning on a clinical outcome training dataset comprising a plurality of training clinical outcomes and a plurality of training characteristics of training patients. The predicted clinical outcome may be obtained at any time before recovery of the patient (based on preoperative, intraoperative, or postoperative imaging data and surveys or any combination thereof). When the predicted clinical outcome is obtained against preoperative imaging data, the outcome model may, for example, be trained to predict a clinical outcome before a clinical intervention or compare alternative intervention strategies. When the predicted clinical outcome is obtained during a medical procedure, the outcome model may, for example, be trained to predict a clinical outcome of an ongoing medical procedure and be used to compare alternatives during the medical procedure. When the predicted clinical outcome is obtained after a medical procedure (but before recovery of the patient), the outcome model may, for example, be used to evaluate the medical procedure, inform the patient about reasonable recovery expectations, and elaborate an effective recovery plan for the patient. Multiple predicted clinical outcomes predicted at different moments may be used to train the same outcome model, or multiple specialized output models may be trained with predicted clinical outcome captures under specific moments or conditions.

The outcome model may be a computational tool designed to predict the likely outcomes of medical interventions, such as surgeries. The outcome model may be trained using machine learning. The dataset used for training the outcome model may consist of historical clinical outcomes and historical characteristics of patients. Historical clinical outcomes may be records of the results of past medical interventions, such as the success rate of surgeries, patient recovery times, complication rates, and any other measurable outcomes relevant to patient health following a medical procedure. Historical characteristics of patients may include demographic information (age, gender, etc.), medical history (previous conditions, surgeries, etc.), and any other relevant characteristics of patients who have undergone medical interventions in the past. The outcome model may use the input from the training dataset to predict the likely outcome of a medical procedure for a new patient. This prediction may be based on the analysis of patterns and correlations found in the historical data, allowing healthcare professionals to make informed decisions about patient care.

After a recovery of the patient from the medical procedure, the predicted clinical outcome may be compared 410 to a measured clinical outcome. Before or during a medical procedure, the outcome model may generate a prediction regarding the likely clinical outcome for the patient based on historical data. This prediction may include various aspects such as the expected recovery time, the likelihood of complications, or the overall success of the procedure. After the medical procedure, the patient may undergo through a recovery period. Upon recovery, the patient's actual outcome may be assessed using measuring various health indicators, patient-reported outcomes, and other metrics relevant to the procedure's success or failure. The predicted outcome made by the model before or during the procedure may then compared to the measured outcome observed after the patient's recovery. Postoperative measurements may include the patient's achieved mobility, any complications that arose during or after the surgery, and an overall prognosis for recovery as observed in clinical follow-ups. The reported outcomes may be quantified based on clinical assessments, patient reports, and objective measurements (e.g., range of motion tests, imaging studies). The measured clinical outcomes may then be compared with the outcomes that were predicted by the model prior to or immediately following the surgery.

Discrepancies between the predicted clinical outcome and the reported outcome may be indicative of corrective elements for adjustment of the outcome model. The comparison may assess the outcome model's accuracy and reliability by evaluating how closely the predicted outcomes align with the actual outcomes experienced by the patient.

The measured clinical outcome may be contributed 420 to the clinical outcome training dataset for continuous improvement thereof. The measured clinical outcome, especially when deviating from the predicted outcome, may be incorporated to the training dataset such that an updated outcome model, trained on the updated dataset, may provide more accurate predictions. The outcome training dataset may serve as a repository of real-world outcomes that can be used to train and refine the outcome models. Adding new patient data to the outcome training dataset may enable the outcome model to continuously learns new cases. Upon enriching training dataset, the outcome model may undergo retraining or fine-tuning processes.

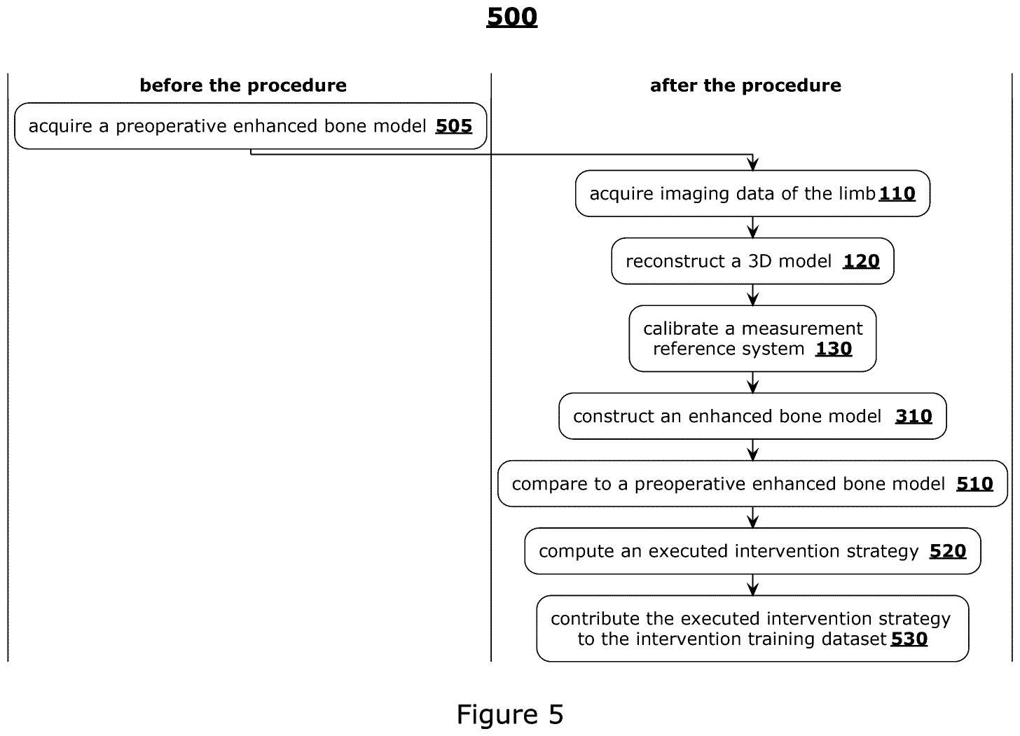

An interpretable 3D model of a limb may be obtained after completion of the medical procedure for training an intervention model. Reference is now made to the drawings in which FIG. 5 depicts a method 500 for obtaining an interpretable 3D model for training an intervention model in accordance with the teachings of the present invention.

An enhanced bone model may be constructed 310 from the interpretable 3D model and a plurality of measurements from the measurement reference system. The enhanced bone model construction 310 may be performed from the postoperative interpretable 3D model and the measurement reference system as described in method 300 hereinabove. The enhanced bone model may be enriched with detailed measurements that reflect the postoperative conditions of the limb.

The enhanced bone model may be compared 510 to a preoperative enhanced bone model. The preoperative enhanced bone model may be acquired 505 from an enhanced bone model constructed from preoperative imaging data and preoperative measurements. The preoperative enhanced bone model may differ from the postoperative enhanced bone model. After the surgery, the new enhanced bone model may be created using the updated imaging data that reflects the current state of the bone and surrounding anatomy. The new enhanced bone model may incorporate any changes made during the surgery, such as alterations to bone structure, the addition of implants, or corrections to alignment. The comparison between the preoperative and postoperative enhanced bone models may involve analyzing the differences between the two models to evaluate how the surgery has altered the bone structure. The comparison may include examining changes in bone geometry, alignment improvements, the positioning of any implants, and the overall anatomical accuracy of the surgical outcome compared to the surgical plan. The comparison may help in assessing whether the surgery achieved its intended objectives, such as correcting deformities, restoring alignment, or properly placing implants.

An executed intervention strategy may be computed 520 from the enhanced bone model and the preoperative enhanced bone model. The executed intervention strategy may refer to the actual surgical actions and decisions made during the operation. The executed intervention strategy may include steps performed by the surgical team to achieve the desired outcomes, including any deviations from the original plan due to intraoperative findings. Computing the executed intervention strategy may involve analyzing the differences between the preoperative and postoperative enhanced bone models to identify what changes were made during the surgery. The comparison may provide insight on changes that were introduced during the medical intervention based on actual findings, judgment, and experience.

The executed intervention strategy may be contributed 530 to an intervention training dataset for continuous improvement thereof. Upon updating the intervention model with the updated intervention training dataset, the contributed executed intervention strategy may provide corrective data or improvements thereto.

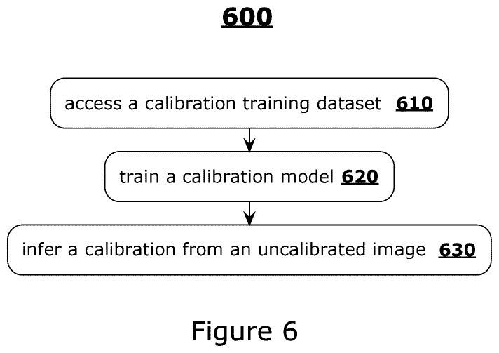

A second aspect of the technique described herein relates to a method, device, and system for calibrating medical imaging data. Reference is now made to the drawings in which FIG. 6 depicts a method 600 for calibrating medical imaging data in accordance with the teachings of the present invention.

A calibration training dataset of calibrated imaging data of a limb may be accessed 610. The calibration training dataset may be a collection of imaging data that has already undergone calibration. Depending on the type of medical procedure, the dataset may specifically include imaging data of a specific limb and its surroundings. The imaging dataset may, for example, relate to images of a lower limb, such as the knee area, from a wide range of patients. The dataset may be stored or located in a manner where it can be retrieved or made available for use. Access 610 may be performed by querying a database or obtaining data from a data repository, or a cloud storage service. The calibrated imaging datasets may be stored in a structured database, ensuring easy retrieval. Metadata describing each dataset (e.g., imaging modality, patient demographics, clinical notes) may also be stored alongside the images. Access 610 to the database may be local or remote across a network or cloud infrastructure.

In embodiments, the calibration training dataset may comprise radiographs, magnetic resonance imaging (MRI) images, ultrasounds, and/or CT scans. The use of radiographs, MRI, ultrasounds, CT scans may depend on the available equipment or the medical procedure. Persons skilled in the art will readily understand that the current disclosure may apply to other types of imaging with the necessary adaptations.

The calibrated imaging data may have been calibrated using auto-calibration algorithms. Auto-calibration may automatically adjust and tunes itself to correct for distortions, inaccuracies, or variations without the need for manual intervention or external reference standards. In the context of medical imaging, auto-calibration may focus on ensuring that images accurately represent the anatomical structures being scanned, despite potential variations in imaging equipment, patient positioning, or other environmental factors that could introduce errors or inconsistencies. Auto-calibration algorithms may be designed to detect issues such as geometric distortions (e.g., caused by the equipment or the way the patient is positioned) and correct them to produce an image that accurately reflects the true dimensions and shapes of the anatomical features. Auto-calibration algorithms may adjust images to correct geometric distortions, ensuring that the proportions and orientations of anatomical structures are accurately represented. Variations in image intensity due to differences in equipment settings or scanning protocols may be standardized through auto-calibration, enhancing the comparability of images taken at different times or with different machines. Auto-calibration may adjust for differences between imaging devices, making it possible to compare images from different sources more reliably.

The calibrated imaging data may be further compensated for geometric distortions and variations in imaging equipment. By compensating for geometric distortions and variations in imaging equipment, the trained model may learn to also compensate for them. The calibration process may address issues such as geometric distortions, variations in imaging equipment, and differences in patient positioning, which may affect the accuracy and consistency of the imaging data. Calibration may involve adjusting the imaging data to correct for geometric distortions, standardizing scaling, and ensure consistency across different imaging modalities and equipment. Calibration may involve aligning the images to a common reference frame or applying corrections based on known calibration phantoms or markers visible in the images. Experts may also annotate the calibrated images with relevant clinical and morphological parameters, such as identifying anatomical landmarks, measuring distances, and noting any pathologies.

A calibration model may be trained 620 with machine learning on the calibration training dataset. The training process may involve providing pairs of uncalibrated samples and calibrated output. A subset of the dataset may be put aside to test the trained calibration model.

A calibration may be inferred 630 from an uncalibrated image data of the limb and the calibration model. The trained calibration model may be capable of applying the learned transformations to new image data. Once trained, the calibration model may be applied to new, uncalibrated imaging data of a patient's limb. The calibration model may process uncalibrated data, applying the learned calibration techniques to correct geometric distortions and standardize a scale. As part of the calibration process, the calibration model may further identify dimensional and morphological parameters within the new imaging data, including detecting and measuring anatomical landmarks, calculating bone lengths and angles, and identifying other features relevant for surgical planning.

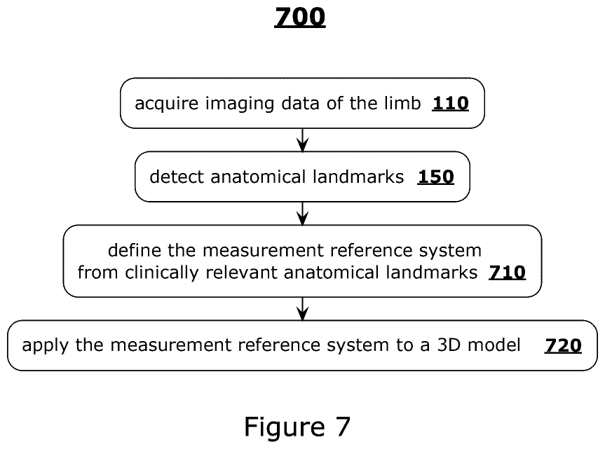

A third aspect of the technique described herein relates to a method, device, and system for establishing a measurement reference system for a medical procedure. Reference is now made to the drawings in which FIG. 7 depicts a method 700 for establishing a measurement reference system for a medical procedure in accordance with the teachings of the present invention.

Imaging data of the limb may be acquired 110. Image data acquisition may be performed in similar ways as previously described hereinabove, for example in method 100.

One or more anatomical landmarks may be detected 150 within the imaging data. The one or more anatomical landmarks may be detected 150 using one or more of an image segmentation, a linear statistical modeling, a non-linear statistical modeling, and a deep learning technique comprising any one of a convolutional neural network (CNN), a recurrent neural network (RNNs), graph neural networks (GNNs) and a transformer networks. Landmark detection may be achieved in various ways, including convolutional neural networks (CNNs) as described hereinabove, for example in method 100.

As also explained hereinabove, an anatomical landmark of the one or more anatomical landmarks may be represented using a geometric shape comprising a least one of a sphere, a cylinder, a cone, an axis, and a plane.

The measurement reference system may be defined 710 from clinically relevant anatomical landmarks of the one or more anatomical landmarks and comprise a set of axes in anatomically interpretable planes. As described hereinabove, an intraoperative measurement reference system on the intraoperative 3D model may be calibrated 130 with a set of axes in clinically interpretable anatomical planes. When multiple interpretable 3D models are used to represent the lower limb, individual reference systems may be provided in addition or in replacement of a common measurement reference system.

The measurement reference system may be applied 720 to a reconstructed 3D model thereby enabling morphological parameters and alignment parameters measurement. As described hereinabove, using these anatomical landmarks, anatomical planes may be defined within the reconstructed 3D model. Common planes used in knee surgery may include the sagittal plane, the coronal (frontal) plan and the transverse (axial) plane. Once the planes are defined, a set of axes may be established within the reconstructed 3D model, thereby obtaining an interpretable 3D model. The axes may be orthogonal to each other and may be aligned with the defined anatomical planes. The axes may serve as reference lines for measuring distances, angles, and alignments within the lower limb. The reference system may be designed to facilitate measurements that are clinically interpretable. Measurements taken using the reference system may be directly related to clinical assessments, decisions, and outcomes. For instance, varus or valgus alignment of the knee, which indicates the inward or outward angulation of the knee, may be precisely assessed using the defined coronal plane. Software processing may be used to overlay the measurement reference system onto the intraoperative 3D model. Real-time visualization and manipulation of the intraoperative 3D models may enable measuring, evaluation, and adjustment of the procedures based on the established reference system. Throughout the surgical procedure, the measurement reference system may be used to monitor changes, make real-time adjustments, and ensure that the surgical intervention aligns with the preoperative planning. For example, the placement of implants may be checked against the reference system to ensure proper alignment and orientation.

The reference frame may be created by identifying specific points or areas of the knee that have clinical significance for knee surgery, such as certain bone points, interfaces, or anatomical structures visible on the imaging data collected during the surgical process. The anatomical landmarks may serve as reference points for constructing a coordinate system or set of axes within the imaging data. The axes may be aligned with the anatomical planes of the knee, such as the sagittal plane (dividing the knee into left and right parts), the coronal plane (dividing the knee into front and back parts), and the axial plane (dividing the knee into upper and lower parts).

Once the anatomical landmarks have been identified and a standardized reference frame has been defined using the surgery imaging data, the reference frame may be applied to a reconstructed 3D model thereby obtaining an interpretable 3D model of the patient's limb. The reconstructed 3D model may be reconstructed 120 from the imaging data and may represent a detailed digital reconstruction of the limb's anatomy, as described in the other methods hereinabove. The standardized reference frame may enable measurements of various morphological (related to the shape and structure) and alignment (related to the positioning and orientation) parameters of the limb. The measurements may include distances between anatomical landmarks, angles of bones relative to each other, the curvature of bone surfaces, and the alignment of the knee joint in three dimensions. The measurements may be used to evaluate the severity of limb conditions, plan corrective measures, and predict the surgery's outcomes.

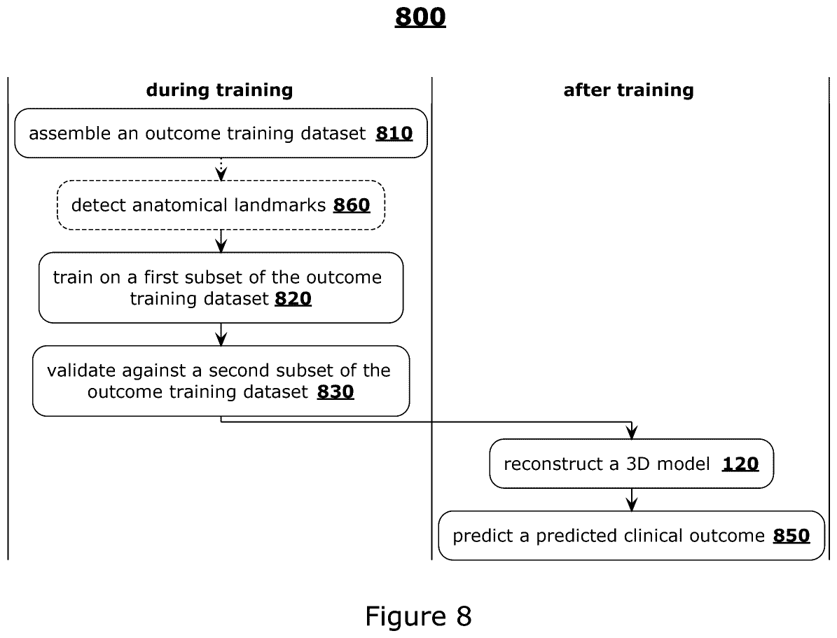

A fourth aspect of the technique described herein relates to a method, device, and system for simulating an intervention strategy in a medical procedure on a limb of a patient. Reference is now made to the drawings in which FIG. 8 depicts a method 800 for simulating an intervention strategy in a medical procedure on a limb of a patient in accordance with the teachings of the present invention.

In embodiments, the intervention strategy may comprise at least one of a prosthetic implant positioning, a meniscal repair, a meniscal resection, a patellar resurfacing, a patellar realignment, a cartilage restoration procedure, a tibial realignment osteotomy, a femoral realignment osteotomy, and a reconstruction of one or more ligament.

During a training phase of an outcome model, an outcome training dataset may be assembled 810. The outcome training dataset may include a plurality of outcome training tuples. Each training tuple may include a training 3D model of a training limb of a training patient, reconstructed from training image data acquired prior to executing a training intervention strategy, the training intervention strategy, a training intervention scenario of the training intervention strategy, a plurality of training patient metadata comprising demographic, anatomical, and physiological parameters, and a measured clinical outcome observed after the medical procedure. The training 3D model may be an interpretable 3D model.

The outcome training dataset may include interpretable 3D models derived from enhanced imaging data, a postoperative 3D model aligned with postoperative imaging data comprising prosthetic placement, a plurality of patient-specific metadata comprising demographic, anatomical, and physiological parameters, and a clinical outcome observed after the knee surgery procedure. The interpretable 3D models may represent limbs of patients and may be constructed from imaging technologies such as MRI, CT scans, or ultrasounds using methods presented hereinabove or similar methods.

The one or more anatomical landmarks from the outcome training dataset may be detected 860 using one or more of an image segmentation, a linear statistical modeling, a non-linear statistical modeling, and a deep learning technique comprising any one of a convolutional neural network (CNN), a recurrent neural network (RNNs), graph neural networks (GNNs) and a transformer networks. Detection 860 of the one or more anatomical landmarks may comprise the use of geometric methods to model the anatomical landmark as one or may geometric shapes, as previously described hereinabove.

The outcome model may be trained 820 with machine learning on a first subset of the outcome training dataset. A specific type of outcome model may be selected based on the nature of the data and the prediction tasks at hand. Given the complexity and high dimensionality of the data involved in knee surgery planning (including interpretable 3D models, patient demographics, and clinical outcomes), deep learning models, particularly convolutional neural networks (CNNs), may be particularly well suited. The deep learning training dataset may be divided into two subsets: one for training the model and another for validating its predictions. The training subset may be used to teach the model to recognize patterns and relationships in the data, while the validation subset may be used to evaluate the model's performance on data it has not seen before. During the training process, the outcome model may be exposed to the training subset of the dataset and learns to associate specific input data (e.g., interpretable 3D models of patient limbs, demographic data) with the desired output (e.g., successful surgical outcomes). The model may adjust the internal parameters of the outcome model to minimize the difference between its predictions and the actual outcomes in the training data. The training process may be iterative, with the model gradually improving its accuracy with each pass through the training data. Throughout training, various techniques may be employed to optimize the model's performance and prevent overfitting (where the model performs well on the training data but poorly on new, unseen data). These techniques may include regularization methods, adjusting the model's architecture, and fine-tuning hyperparameters.

An output of the outcome model may be validated 830 against a second subset of the outcome training dataset. The outcome model's predictions may be regularly validated against the validation subset of the dataset to ensure that the outcome model is learning to generalize well to new data. The outcome of the training process may be an outcome model that has learned the complex relationships between the anatomical features, patient-specific metadata, and clinical outcomes contained in the deep learning training dataset. The outcome model may then be used to simulate intervention strategies for new patients, providing recommendations for surgical planning based on predicted outcomes.

After the training phase of the outcome model, an interpretable 3D model of the limb of the patient may be reconstructed 120 from image data acquired 110 prior to executing the intervention strategy.

The interpretable 3D models may provide a detailed view of the patient's anatomy before the surgery. After the surgery, new imaging data is captured to assess the outcome, including an executed intervention strategy. The postoperative imaging data may be used to create updated 3D models that reflect the immediate results of the surgery. Aligning these models with the original preoperative 3D models may for a direct comparison of surgical outcomes. The deep learning training dataset may encompass information specific to each patient, including but not limited to age, gender, body mass index (BMI), pre-existing health conditions, and specific anatomical and physiological characteristics relevant to knee surgery. The deep learning training dataset may also include detailed records of clinical outcomes observed after the surgeries. The deep learning training dataset can include aspects such as the degree of pain relief achieved, the range of motion restored, the speed of recovery, and any complications that arose.

A predicted clinical outcome may be predicted 850 from the outcome model, the interpretable 3D model, the intervention strategy, an intervention scenario of the intervention strategy, and a plurality of patient metadata.

In the case of a prosthetic implant positioning, the simulation may allow a virtual assessment of how the prosthesis may be positioned in the patient's knee before the surgery, considering the patient's specific enhanced bone model and the data learned by the trained outcome model to optimize the placement and orientation of the prosthesis. The simulation may enhance surgical planning anticipating the positioning of the prosthesis, which may lead to better postoperative outcomes for the patient. In certain intervention strategies, no prothesis is used and the intervention is focused on in-situ modifications of one or more physiological structures of the patient (e.g., bone, ligament, or the likes). Persons skilled in the art will readily recognize that different intervention strategies may be considered for different types of knee surgeries.

The outcome model may be obtained through deep learning by following a multi-step process. Large datasets of medical images may thereby be leveraged and information about successful surgeries may be used in the context of deep learning algorithms to recognize optimal prosthesis alignments. The first step may involve gathering a substantial dataset of preoperative and postoperative imaging data (such as radiographs, MRI images, ultrasounds, and CT scans) from patients who have undergone knee surgery. The dataset of preoperative and postoperative imaging data may include a wide variety of knee anatomies, conditions, and outcomes to ensure the model can learn from diverse examples. Information on the specific prosthesis used, the alignment strategy employed, and the postoperative outcomes (including recovery speed, any complications, and long-term functionality) may also be collected. The dataset may need to be processed to be useful for training a deep learning model. Preprocessing tasks may include image normalization, contrast enhancement, and the annotation of key anatomical landmarks. For alignment parameters specifically, annotations may include the desired position and orientation of the prosthesis within the knee joint. With the processed dataset, a deep learning model such as a CNN may be trained. The deep learning model may be presented with preoperative images and the corresponding postoperative outcomes, learning to recognize patterns and features associated with successful prosthesis placements. The training process may involve adjusting the model's internal parameters to minimize the difference between its predictions (e.g., recommended prosthesis alignment) and the actual successful outcomes in the training data. Techniques such as transfer learning, where a pre-trained model is fine-tuned with the specific dataset may accelerate the training process. The trained model may then be validated and tested using a separate set of data not seen by the model during training. Validation may involve comparing the model's prosthesis placement recommendations with the actual successful placements used in surgeries. Based on the performance in the testing phase, the model may be further refined and adjusted. Adjustments may involve additional training rounds, tweaking the model architecture, or incorporating more diverse data into the training set.

The comparative analysis may include impact of different surgical strategies on clinical outcomes. Historical postoperative outcome data may include information on the outcomes of knee surgeries conducted in the past and may include details such as the recovery time, any complications that arose, the long-term success of the surgery (e.g., improvement in mobility, pain reduction), and the overall satisfaction of the patients. Historical data may serve as a reference point for understanding the range of possible outcomes following knee surgery and the factors that may influence these outcomes. The characteristics of the patient may include a wide array of patient-specific information, such as age, sex, body mass index (BMI), underlying health conditions (e.g., diabetes, osteoporosis), lifestyle factors (e.g., activity level), and the specific anatomy of the patient's knee (obtained from preoperative imaging data). The characteristics of the patient may influence the outcome of a surgery.

The choice of algorithm may depend on the complexity of the data and the specific outcomes of interest. To refine the predictive outcome model, feature selection techniques may be employed to identify which parameters most significantly influence the clinical outcomes. Feature selection may improve accuracy of the trained outcome model and interpretability of the outcome. The trained outcome model may be validated using a separate portion of the historical dataset not involved in the training process. Validation may assess the outcome model's accuracy in predicting outcomes and helps identify any biases or overfitting issues. Once validated, the outcome model may be applied to the integrated dataset created from the patient's postoperative bone 3D model. The trained outcome model may compute the likelihood of achieving specific mobility thresholds, assesses the risk of potential complications, and estimates the overall prognosis for recovery based on the patterns learned during training.

The methods described herein may be performed by devices and systems. Reference is now made to the drawings in which FIG. 9 shows a logical modular representation of an exemplary system 2000 comprising an assisting device 2100. The device 2100 comprises a memory module 2160, a processor module 2120, an analysis module 2130 and a network interface module 2170. The assisting device 2100 may also include a display module 2150.

In accordance with the first aspect, the system 2000 may comprise an imaging capture module 2600 and the assisting device may comprise one or more processors 2120. The imaging capture module 2600 may acquire imaging data of a limb. The one or more processors 2120 may reconstruct a reconstructed 3D model of the limb from the imaging data and calibrate a measurement reference system on the reconstructed 3D model, comprising a set of axes in clinically interpretable anatomical planes, thereby obtaining the interpretable 3D model. In embodiments, the assisting device 2100 may comprise a display device 2150 to display reports.

The imaging capture device 2600 may be connected to the assisting device 2100 across a network interface module 2170 and 2170′.

In accordance with the second aspect, the assisting device 2100 may comprise one or more processors 2120. The one or more processors 2120 may access a calibration training dataset of calibrated imaging data of a limb. The one or more processors 2120 may further train a calibration model with machine learning on the calibration training dataset. The one or more processors 2120 may further infer a calibration from an uncalibrated image data of the limb and the calibration model.

In accordance with the third aspect, the system 2000 may comprise an imaging capture module 2600 and the assisting device 2100 may comprise one or more processors 2120. The imaging capture module may acquire imaging data of the limb. The one or more processors 2120 may detect one or more anatomical landmarks within the imaging data. The one or more processors 2120 may further define the measurement reference system from clinically relevant anatomical landmarks of the one or more anatomical landmarks, the measurement reference system comprising a set of axes in anatomically interpretable planes. The one or more processors 2120 may further apply the measurement reference system to a 3D model thereby enabling morphological parameters and alignment parameters measurement.

In accordance with the fourth aspect, a processor module 2120 of an assisting device 2100 may comprise one or more training processors and an analysis module 2130 of the assisting device 2100 may comprise one or more inferring processors. During a training phase of an outcome model, the one or more training processors 2120 may assemble an outcome training dataset comprising a plurality of outcome training tuples. Each training tuples may comprise a training 3D model of a training limb of a training patient, reconstructed from training image data acquired prior to executing a training intervention strategy, the training intervention strategy, a training intervention scenario of the training intervention strategy, a plurality of train patient metadata comprising demographic, anatomical, and physiological parameters, and a measured clinical outcome observed after the medical procedure. The one or more training processors 2120 may further train the outcome model with machine learning on a first subset of the outcome training dataset. The one or more training processors 2120 may further validate an output of the outcome model against a second subset of the outcome training dataset. After the training phase of the outcome model, the one or more inferring processors 2130 may reconstruct a 3D model of the limb of the patient from image data acquired 110 prior to executing the intervention strategy. The one or more inferring processors 2130 may further predict a predicted clinical outcome from the outcome model, the 3D model, the intervention strategy, an intervention scenario of the intervention strategy, and a plurality of patient metadata.

The system 2000 may comprise a storage system 2300 for storing and accessing long-term (i.e., non-transitory) data and may further log data while the assisting device 2100 is being used. FIG. 1 shows examples of the storage system 2300 as a distinct database system 2300A, a distinct module 2300C of the assisting device 2100 or a sub-module 2300B of the memory module 2160 of the assisting device 2100. The storage system 2300 may be distributed over different systems A, B, C. The storage system 2300 may comprise one or more logical or physical as well as local or remote hard disk drive (HDD) (or an array thereof). The storage system 2300 may further comprise a local or remote database made accessible to the assisting device 2100 by a standardized or proprietary interface or via the network interface module 2170.

The network interface module 2170 represents at least one physical interface that can be used to communicate with an imaging capture device 2600. The network interface module 2170 may be made visible to the other modules of the imaging capture device 2600 through one or more logical interfaces. The actual stacks of protocols used by the physical network interface(s) and/or logical network interface(s) 2172-2178 of the network interface module 2170 do not affect the teachings of the present invention.

The processor module 2120 may represent a single processor with one or more processor cores or an array of processors, each comprising one or more processor cores. The memory module 2160 may comprise various types of memory (different standardized or kinds of Random Access Memory (RAM) modules, memory cards, Read-Only Memory (ROM) modules, programmable ROM, etc.).

A bus 2180 is depicted as an example of means for exchanging data between the different modules of the assisting device 2100. The teachings presented herein are not affected by the way the different modules exchange information. For instance, the memory module 2160 and the processor module 2120 could be connected by a parallel bus, but could also be connected by a serial connection or involve an intermediate module (not shown) without affecting the teachings of the present invention.

An analysis module 2130 provides prediction-related services to the assisting device 2100, which will be described in more details hereinbelow.

The variants of processor module 2120, memory module 2160 and network interface module 2170 usable in the context of the present invention will be readily apparent to persons skilled in the art. Likewise, even though explicit mentions of the analysis module 2130, the memory module 2160, the display module 2150 and/or the processor module 2120 are not made throughout the description of the present examples, persons skilled in the art will readily recognize when such modules are used in conjunction with other modules of the assisting device 2100 to perform routine as well as innovative elements presented herein.

Various network links may be implicitly or explicitly used in the context of the present invention. While a link may be depicted as a wireless link, it could also be embodied as a wired link using a coaxial cable, an optical fiber, a category 5 cable, and the like. A wired or wireless access point (not shown) may be present on the link between. Likewise, any number of routers (not shown) may be present and part of the link, which may further pass through the Internet.

While illustrative and presently preferred embodiment(s) of the invention have been described in detail hereinabove, it is to be understood that the inventive concepts may be otherwise variously embodied and employed and that the appended claims are intended to be construed to include such variations except insofar as limited by the prior art.

1. A method for obtaining an interpretable 3D model for a medical procedure on a limb, the method comprising:
acquiring imaging data of the limb;
reconstructing a reconstructed 3D model of the limb from the imaging data; and
calibrating a measurement reference system on the reconstructed 3D model, the measurement reference system comprising a set of axes in clinically interpretable anatomical planes, thereby obtaining the interpretable 3D model.

2.-4. (canceled)

5. The method of claim 1, further comprising:
detecting one or more anatomical landmarks from the imaging data, and wherein reconstructing the reconstructed 3D model is performed considering the one or more anatomical landmarks,
the one or more anatomical landmarks are detected using one or more of an image segmentation, a linear statistical modeling, a non-linear statistical modeling, and a deep learning technique comprising any one of a convolutional neural network (CNN), a recurrent neural network (RNNs), graph neural networks (GNNs) and a transformer networks; and
an anatomical landmark of the one or more anatomical landmarks is represented using a geometric shape comprising a least one of a sphere, a cylinder, a cone, an axis, and a plane.

6. (canceled)

7. The method of claim 1, further comprising enhancing the imaging data through one or more image processing techniques, wherein the one or more image processing techniques comprise an image enhancement model trained with a plurality of enhancement image pairs, each comprising an information poor image and information-rich image.

8.-9. (canceled)

10. The method of claim 1, further comprising:
monitoring the medical procedure, the monitoring being performed by:
acquiring, in real-time, updated imaging data of a current state of the limb;
registering the updated imaging data for the measurement reference system;
updating the interpretable 3D model with the updated imaging data into an updated 3D model;
comparing the updated 3D model with a planned intervention strategy; and
reporting, in real-time, discrepancies between the current state of the limb and the planned intervention strategy.

11. The method of claim 10, further comprising:
constructing an enhanced bone model from the interpretable 3D model and a plurality of measurements from the measurement reference system; and
comparing the enhanced bone model with the planned intervention strategy.

12.-14. (canceled)

15. The method of claim 1, further comprising:
predicting a clinical outcome of the medical procedure on a patient, the predicting being performed by:
constructing an enhanced bone model from the interpretable 3D model and a plurality of measurements from the measurement reference system;
generating one or more intervention strategies from an intervention scenario and the enhanced bone model; and
predicting the predicted clinical outcome for at least one intervention strategy of the one or more intervention strategies and a plurality of characteristics of the patient.

16. The method of claim 15, wherein the plurality of measurements comprises at least one of a plurality of morphological parameters, a plurality of alignment parameters and a plurality of kinematics parameters.

17. The method of claim 15, wherein the one or more intervention strategies comprises at least one of a prosthetic implant positioning, a meniscal repair, a meniscal resection, a patellar resurfacing, a patellar realignment, a cartilage restoration procedure, a tibial realignment osteotomy, a femoral realignment osteotomy, and a reconstruction of one or more ligament.

18. The method of claim 15, further comprising:
computing an outcome score of the at least one intervention strategy; and
sorting the predicted clinical outcome according to the outcome score.

19. (canceled)

20. The method of claim 15, the method further comprising recording one or more joint kinematics parameters and wherein constructing the enhanced bone model is performed considering the one or more joint kinematics parameters, the one or more joint kinematics parameters comprising at least one of a rotation, a translation, a weight-bearing gap measurement, a free gap measurement, a manually stressed gap measurement, a mechanically stressed gap measurement, and a contact point.

21. (canceled)

22. The method of claim 15, wherein predicting the predicted clinical outcome is performed using an outcome model trained with machine learning on a clinical outcome training dataset comprising a plurality of training clinical outcomes and a plurality of training characteristics of training patients, the method, further comprising:
after a recovery of the patient from the medical procedure, training the outcome model by:
comparing the predicted clinical outcome to a measured clinical outcome; and
contributing the measured clinical outcome to the clinical outcome training dataset for continuous improvement thereof.

23. (canceled)

24. The method of claim 1, when performed after completion of the medical procedure, the method further comprising:
training an intervention model by:
constructing an enhanced bone model from the interpretable 3D model and a plurality of measurements from the measurement reference system;
comparing the enhanced bone model to a preoperative enhanced bone model;
computing an executed intervention strategy from the enhanced bone model and the preoperative enhanced bone model; and
contributing the executed intervention strategy to an intervention training dataset for continuous improvement thereof.

25. A method for calibrating medical imaging data, the method comprising:
accessing a calibration training dataset of calibrated imaging data of a limb;
training a calibration model with machine learning on the calibration training dataset; and
inferring a calibration from an uncalibrated image data of the limb and the calibration model,
wherein the calibration training dataset comprises at least one of a radiograph, a magnetic resonance imaging (MRI) image, an ultrasound, and a computed tomography (CT) scan.

26. (canceled)

27. The method of claim 25, wherein the calibrated imaging data has been calibrated using auto-calibration algorithms and wherein the calibrated imaging data is further compensated for geometric distortions and variations in imaging equipment.

28. The method of claim 27, wherein the calibrated imaging data is further compensated for geometric distortions and variations in imaging equipment.

29. A method for establishing a measurement reference system for a medical procedure, the method comprising:
acquiring imaging data of a limb;
detecting one or more anatomical landmarks within the imaging data;
defining the measurement reference system from clinically relevant anatomical landmarks of the one or more anatomical landmarks, the measurement reference system comprising a set of axes in anatomically interpretable planes; and
applying the measurement reference system to a reconstructed 3D model thereby enabling morphological parameters and alignment parameters measurement.

30. The method of claim 29, wherein:
detecting the one or more anatomical landmarks comprises one or more of an image segmentation, a linear statistical modeling, a non-linear statistical modeling, and a deep learning technique comprising any one of a convolutional neural network (CNN), a recurrent neural network (RNNs), graph neural networks (GNNs) and a transformer networks; and
an anatomical landmark of the one or more anatomical landmarks is represented using a geometric shape comprising a least one of a sphere, a cylinder, a cone, an axis, and a plane.

31. A method for simulating an intervention strategy in a medical procedure on a limb of a patient, the method comprising:
during a training phase of an outcome model:
assembling an outcome training dataset comprising a plurality of outcome training tuples, each comprising:
a training interpretable 3D model of a training limb of a training patient, reconstructed from training image data acquired prior to executing a training intervention strategy;
the training intervention strategy;
a training intervention scenario of the training intervention strategy;
a plurality of training patient metadata comprising demographic, anatomical, and physiological parameters; and
a measured clinical outcome observed after the medical procedure;
training the outcome model with machine learning on a first subset of the outcome training dataset; and
validating an output of the outcome model against a second subset of the outcome training dataset;
after the training phase of the outcome model:
reconstructing an interpretable 3D model of the limb of the patient from image data acquired prior to executing the intervention strategy; and
predicting a predicted clinical outcome from the outcome model, the interpretable 3D model, the intervention strategy, an intervention scenario of the intervention strategy, and a plurality of patient metadata.

32. The method of claim 31, further comprising:
detecting one or more anatomical landmarks from the outcome training dataset comprises one or more of an image segmentation, a linear statistical modeling, a non-linear statistical modeling, and a deep learning technique comprising any one of a convolutional neural network (CNN), a recurrent neural network (RNNs), graph neural networks (GNNs) and a transformer networks; and
representing an anatomical landmark of the one or more anatomical landmarks using a geometric shape comprising a least one of a sphere, a cylinder, a cone, an axis, and a plane.

33. The method of claim 31, wherein the intervention strategy comprises at least one of a prosthetic implant positioning, a meniscal repair, a meniscal resection, a patellar resurfacing, a patellar realignment, a cartilage restoration procedure, a tibial realignment osteotomy, a femoral realignment osteotomy, and a reconstruction of one or more ligament.

34.-66. (canceled)
